(19) United States
(12) Patent Application Publication
(10) Pub. No.: US 2025/0339458 A1
(43) Pub. Date: Nov. 6, 2025

(54) USES OF MODIFIED RNA ENCODING RETINALDEHYDE DEHYDROGENASE

(71) Applicant: President and Fellows of Harvard College, Cambridge, MA (US)

(72) Inventors: Ulrich H. von Andrian, Cambridge, MA (US); Bruno Raposo, Cambridge, MA (US)

(73) Assignee: President and Fellows of Harvard College, Cambridge, MA (US)

(21) Appl. No.: 19/273,379

(22) Filed: Jul. 18, 2025

Related U.S. Application Data
(63) Continuation of application No. 17/284,212, filed on Apr. 9, 2021, now abandoned.
(60) Provisional application No. 62/743,943, filed on Oct. 10, 2018.

(51) Int. Cl. A61K 31/7115; A61K 38/44; A61K 39/00

(52) U.S. Cl. CPC A61K 31/7115; A61K 38/44; A61K 39/00; A61K 2039/53; A61K 2039/541; A61K 2039/545; A61K 2039/55561; A61K 2039/70; C12Y 102/01036

(57) ABSTRACT

Some aspects of this disclosure provide modified mRNA (modRNA) encoding retinaldehyde dehydrogenase (RALDH) enzyme, in addition to methods of synthesis, administration, use, and treatment. In some embodiments, the modRNA may be used in a vaccine to treat infections (e.g., mucosal infections) and/or cancers (e.g., mucosal cancers).

### RELATED APPLICATIONS

This application claims priority under 35 U.S.C. § 119(e) to U.S. provisional application number, U.S. Ser. No. 62/743,943, filed Oct. 10, 2018, which is incorporated herein by reference.

### BACKGROUND OF THE INVENTION

The origin and development of vaccines was a turning point in human history and the permanent fight against microbes. Today, vaccines are the most cost-effective way to save lives. Most current vaccines are administered by injection through the skin, which often results in weak or no immune protection at mucosal sites. Because the major point of entry for many human pathogens occurs at gastrointestinal (e.g., polio virus, E. coli, Shigella, V. cholera, HIV-1), respiratory (e.g. influenza virus, M. tuberculosis, adenovirus), or genital (Chlamydia, HIV-1, HPV) mucosal surfaces, protective immunity against mucosal pathogens requires the development of vaccine strategies capable of inducing mucosal immune responses (1, 2). For instance, diarrheal diseases constitute the second leading cause of death (after pneumonia) in young children of developing countries (3), and the design of tailored vaccines to the pathogen(s) and site of infection constitute a great challenge.

Priming of adaptive immune responses, particularly by dendritic cells (DCs), in specific mucosal sites determines subsequent homing of antigen-specific T and B cells to the mucosal source tissue, as well as to other mucosal tissues (4, 5). Therefore, in order to target the intestinal mucosa, most vaccine formulations are designed for oral administration. However, oral vaccines require high dosages of antigen to induce an immune response due to poor antigen stability in the harsh conditions of the gastrointestinal tract, and because a tolerogenic, rather than immunogenic, response often results from oral antigen exposure (6). Moreover, despite the efficacy of oral vaccines in developed countries, their efficacy in developing countries is often unsatisfactory. Nutritional status, ongoing persistent infections with helminths and other parasites, and the intestinal microbiota are thought to play a major role in the vaccines' differential efficiency (7).

Likewise, vaccines targeting memory responses in the uterine or vaginal mucosa require the generation of tissue resident memory cells (5). In such cases, uterine vaccination strategies may generate local immunity, but clinical translation has been found challenging (8).

Thus, there is a need to develop robust parenteral vaccine formulations that are efficiently targeted to mucosal tissues and possess mucosal imprinting properties.

### SUMMARY OF THE INVENTION

Provided herein are modified polynucleotides encoding retinaldehyde dehydrogenase (RALDH). RALDH was selected due to its ability to indirectly upregulate mucosal homing receptors on activated lymphocytes, resulting in a targeted antigen-specific response. Therefore, the modified polynucleotides may be used, for example, as components of vaccines. As described herein, vaccines comprising modified polynucleotides encoding RALDH may be used, for example, to target mucosal tissues, leading to the generation of antigen-specific immunity in said tissues. In one embodiment, the modified polynucleotides, e.g., mRNA, may comprise at least one pseudouridine in place of a uridine and/or at least one 5-methylcytosine in place of a cytosine. Other modifications, as described herein, are also possible. The modified polynucleotides can form the basis of new parenteral vaccine formulations that target mucosal tissues, bypassing the tolerogenic effects commonly associated with mucosal vaccinations as well as the toxicity mediated by other molecules, which also have mucosal imprinting properties, such as all-trans retinoic acid (ATRA). The vaccine, which can, in some embodiments, induce mucosal receptors on antigen-specific T cells, may be used in cancer immunotherapy, particularly in regard to tumors developing at mucosal surfaces.

Provided herein are novel modified polynucleotides (e.g., mRNA) encoding a RALDH protein. The invention, in some aspects, includes a variety of vaccines comprising the modified polynucleotides (e.g, mRNA) described herein. The vaccines may be used, for example, to treat infections (e.g., mucosal infections) or cancers (e.g., mucosal cancers). In some instances, the modified polynucleotide may be used in a tolerogenic vaccine. Another aspect of the invention provides a kit comprising the modified polynucleotide and/or the vaccine described herein.

The modified polynucleotides (e.g., mRNA), in some embodiments, comprise an open reading frame (ORF) encoding a retinaldehyde dehydrogenase (RALDH) protein (e.g., human RALDH), wherein at least one uridine is pseudouridine, and/or at least one cytosine is 5-methylcytosine. Other modifications, as described herein, are also contemplated.

In some embodiments, the RALDH protein is selected from the group consisting of: retinaldehyde dehydrogenase 1 (RALDH1) protein, retinaldehyde dehydrogenase 2 (RALDH2) protein, and retinaldehyde dehydrogenase 3 (RALDH3) protein. In one embodiment, the RALDH protein is RALDH2. In an embodiment, the RALDH protein is a human RALDH protein or variant thereof. Exemplary amino acid and nucleotide sequences of RALDH isoforms are provided herein.

In some embodiments, the open reading frame encodes two RALDH proteins selected from the group consisting of: retinaldehyde dehydrogenase 1 (RALDH1) protein, retinaldehyde dehydrogenase 2 (RALDH2) protein, and retinaldehyde dehydrogenase 3 (RALDH3) protein. In another embodiment, the open reading frame encodes retinaldehyde dehydrogenase 1 (RALDH1) protein, retinaldehyde dehydrogenase 2 (RALDH2) protein, and retinaldehyde dehydrogenase 3 (RALDH3) protein.

In another aspect, the invention provides a vaccine comprising at least one antigen, and at least one modified ribonucleic acid (e.g., mRNA) polynucleotide comprising an open reading frame (ORF) encoding a retinaldehyde dehydrogenase (RALDH) protein.

In some embodiments, the vaccine described herein further comprises an adjuvant (e.g., alum, AS03, AS04, MF59, or TLR agonists). In other embodiments, the vaccine described herein further comprises retinal, retinol, β-carotene, or a combination thereof.

The antigen may be, for example, a polynucleotide, protein, peptide, plasmid, virus, viral fragment, bacteria, bacterial fragment, fungi, fungal fragments and conjugate.

In some embodiments, the vaccine described herein is a mucosal vaccine. In such vaccines, the antigen may be, for example, a viral or a bacterial pathogen, or a combination thereof.

In other embodiments, the vaccine may be a cancer vaccine, and the antigen may be a tumor antigen, e.g., a mucosal tumor antigen. Exemplary mucosal tumor antigens include guanylyl cyclase C, sucrose isomaltase, CDX1, CDX2, mammoglobulin, small breast epithelial mucin, RAGE antigen, MUC1, and neoantigens. In some embodiments, the mucosal tumor antigen is associated with a mucosal cancer selected from the group consisting of colon cancers, head and neck squamous cell carcinomas, lung cancers, cervical cancers, and pancreatic cancers.

The vaccine may be formulated in a variety of different ways, for example, as a nanoparticle, microparticle, liposome, or hydrogel. In one particular example, the vaccine is formulated as a cationic lipid nanoparticle. The vaccine may further comprise a pharmaceutically acceptable excipient.

In yet another aspect, the invention provides a tolerogenic vaccine. Tolerogenic vaccines are used, for example, to reduce an immune response (induce tolerance) in the face of a pathological or unwanted activation of the normal immune response, which occurs, for example in autoimmune disorders. The tolerogenic vaccine, in one aspect, comprises an antigen, an immunomodulatory agent, and at least one modified messenger ribonucleic acid (mRNA) polynucleotide comprising an open reading frame (ORF) encoding a retinaldehyde dehydrogenase (RALDH) protein. Exemplary immunomodulatory agents include, but are not limited to, mTOR inhibitors, HDAC inhibitors, MHC-peptide complexes, and antigen-laden erythrocytes.

The vaccines or pharmaceutical compositions thereof described herein may be used in methods to induce antigen-specific immune responses (e.g., a T cell response or a B cell response), for example, in the mucosal tissues of a subject. Depending on the content of the vaccine of pharmaceutical composition thereof, it may be used to immunize a subject against a pathogen (e.g., a mucosal pathogen). The vaccines or pharmaceutical compositions thereof may also be used to treat an infection (e.g., a mucosal infection) or a cancer (e.g., a mucosal cancer) in a subject.

The vaccines or pharmaceutical compositions thereof may be administered parenterally, for example, by subcutaneous administration or intramuscular administration, or orally. The vaccine or pharmaceutical composition thereof may be administered as a single dose, or as a single dose followed by one or more subsequent booster doses.

Another aspect of the invention provides a kit comprising: a polynucleotide described herein; a pharmaceutically acceptable excipient; a container; and instructions for using the kit. Further kits comprising any one of the vaccines or pharmaceutical compositions thereof are also described herein.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

FIGS. 1A-1D demonstrate that modRNA-RALDH2 induces the expression of gut-homing receptors on antigen-specific lymphocytes. FIG. 1A. Bone marrow-derived dendritic cells were cultured for 48 hours in the presence of 10 nM ATRA (ATRA), 20 μg modRNA-RALDH2 (modRNA), 20 μg modRNA-RALDH2 plus 1 μM retinol (modRNA+retinol), or left untouched (untreated). The expression of RALDH2 was detected by Western blot using a recombinant human RALDH2 sequence with an N-terminal His-tag as a positive control protein. FIGS. 1B-1C. Purified ovalbumin-specific transgenic CD8⁺ T cells (OT-I) were cultured with wild-type splenocytes (APCs) and the indicated supplement. Upregulation of α4β7 and CCR9 was determined using flow cytometry (FIG. 1B) and represented as the geometric mean of the mean fluorescent intensity (GeoMFI; FIG. 1C). FIG. 1D. Wild-type C57BL/6J mice were randomly divided into groups (each group housed separately) and immunized subcutaneously at day 0 with 100 μg ovalbumin (Ova) and 5 μM CpG class B. At days 0 and 2, the corresponding group of mice received 5 mM ATRA, 5 mM retinol, 50 μg mod RNA, or 50 μg mod RNA plus 5 mM retinol, administered subcutaneously at the same site of injection as the Ova/CpG. Fecal samples were collected throughout the duration of the experiment, and titers of anti-Ova IgA-specific antibodies were assessed. All mice were orally challenged by gavage with 10 mg Ova at day 57.

### DEFINITIONS

As used herein and in the claims, the singular forms “a,” “an,” and “the” include the singular and the plural reference unless the context clearly indicates otherwise. Thus, for example, a reference to “an agent” includes a single agent and a plurality of such agents.

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, RNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated.

The term “modified polynucleotide” (e.g., modified RNA, “modRNA”), as used herein, refers to a polynucleotide (e.g., DNA, RNA) that comprises at least one modified nucleotide. For example, the polynucleotide may comprise any of the nucleoside analogs, chemically modified bases, biologically modified bases, intercalated bases, modified sugars, isomers, and/or modified phosphate groups described herein.

The term “open reading frame” (ORF), as used herein, refers to a continuous stretch of RNA beginning with a start codon (e.g., AUG) and ending with a stop codon (e.g., UAA, UAG, UGA) that encodes a protein. In some embodiments, the protein encoded by the ORF is a retinaldehyde dehydrogenase (RALDH) protein.

In some embodiments, the ORF is codon-optimized. As used herein, “codon-optimized polynucleotide” refers to a polynucleotide that comprises codons that do not match those of the wild-type polynucleotide, but that do not alter the translated amino acid sequence of the encoded protein. The optimized codons can be used, for example, to increase mRNA stability, reduce secondary structures, minimize tandem repeat codons or base runs (which may impair gene construction or expression), manipulate transcriptional and translational control regions, add or delete protein trafficking sequences, insert, delete, or shuffle protein domains, add or delete restriction sites, or match codon frequencies in target and host organisms (for proper folding and secondary structure). Codon optimization tools, algorithms and services are known in the art, and non-limiting examples include services from GeneArt (Life Technologies), DNA2.0 (Menlo Park CA) and/or proprietary methods.

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. A protein may comprise different domains, for example, a nucleic acid binding domain. In some embodiments, a protein is in a complex with, or is in association with, a nucleic acid, e.g., RNA. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

In some embodiments, the protein is a RALDH protein. There are three splice variants (isoforms) of RALDH, termed RALDH1, RALDH2, and RALDH3. RALDH proteins are enzymes that catalyze the synthesis of retinoic acid (RA) from retinaldehyde, β-carotene, and vitamin A (retinol). The amino acid sequences of each isoform are given below:

RALDH1 (H. sapiens) - NP_000680.2
(SEQ ID NO: 4)
MSSSGTPDLPVLLTDLKIQYTKIFINNEWHDSVSGKKFPVFNPATEEELC
QVEEGDKEDVDKAVKAARQAFQIGSPWRTMDASERGRLLYKLADLIERDR
LLLATMESMNGGKLYSNAYLNDLAGCIKTLRYCAGWADKIQGRTIPIDGN
FFTYTRHEPIGVCGQIIPWNFPLVMLIWKIGPALSCGNTVVVKPAEQTPL
TALHVASLIKEAGFPPGVVNIVPGYGPTAGAAISSHMDIDKVAFTGSTEV
GKLIKEAAGKSNLKRVTLELGGKSPCIVLADADLDNAVEFAHHGVFYHQG
QCCIAASRIFVEESIYDEFVRRSVERAKKYILGNPLTPGVTQGPQIDKEQ
YDKILDLIESGKKEGAKLECGGGPWGNKGYFVQPTVFSNVTDEMRIAKEE
IFGPVQQIMKFKSLDDVIKRANNTFYGLSAGVFTKDIDKAITISSALQAG
TVWVNCYGVVSAQCPFGGFKMSGNGRELGEYGFHEYTEVKTVTVKISQKN
S
RALDH2 (H. sapiens) - NP_003879.2
(SEQ ID NO: 5)
MTSSKIEMPGEVKADPAALMASLHLLPSPTPNLEIKYTKIFINNEWQNSE
SGRVFPVYNPATGEQVCEVQEADKADIDKAVQAARLAFSLGSVWRRMDAS
ERGRLLDKLADLVERDRAVLATMESLNGGKPFLQAFYVDLQGVIKTFRYY
AGWADKIHGMTIPVDGDYFTFTRHEPIGVCGQIIPWNFPLLMFAWKIAPA
LCCGNTVVIKPAEQTPLSALYMGALIKEAGFPPGVINILPGYGPTAGAAI
ASHIGIDKIAFTGSTEVGKLIQEAAGRSNLKRVTLELGGKSPNIIFADAD
LDYAVEQAHQGVFFNQGQCCTAGSRIFVEESIYEEFVRRSVERAKRRVVG
SPFDPTTEQGPQIDKKQYNKILELIQSGVAEGAKLECGGKGLGRKGFFIE
PTVFSNVTDDMRIAKEEIFGPVQEILRFKTMDEVIERANNSDFGLVAAVF
TNDINKALTVSSAMQAGTVWINCYNALNAQSPFGGFKMSGNGREMGEFGL
REYSEVKTVTVKIPQKNS
RALDH3 (H. sapiens) - NP_000684.2
(SEQ ID NO: 6)
MATANGAVENGQPDRKPPALPRPIRNLEVKFTKIFINNEWHESKSGKKFA
TCNPSTREQICEVEEGDKPDVDKAVEAAQVAFQRGSPWRRLDALSRGRLL
HQLADLVERDRATLAALETMDTGKPFLHAFFIDLEGCIRTLRYFAGWADK
IQGKTIPTDDNVVCFTRHEPIGVCGAITPWNFPLLMLVWKLAPALCCGNT
MVLKPAEQTPLTALYLGSLIKEAGFPPGVVNIVPGFGPTVGAAISSHPQI
NKIAFTGSTEVGKLVKEAASRSNLKRVTLELGGKNPCIVCADADLDLAVE
CAHQGVFFNQGQCCTAASRVFVEEQVYSEFVRRSVEYAKKRPVGDPFDVK
TEQGPQIDQKQFDKILELIESGKKEGAKLECGGSAMEDKGLFIKPTVFSE
VTDNMRIAKEEIFGPVQPILKFKSIEEVIKRANSTDYGLTAAVFTKNLDK
ALKLASALESGTVWINCYNALYAQAPFGGFKMSGNGRELGEYALAEYTEV
KTVTIKLGDKNP

The term “untranslated region” (UTR), as used herein, refers to a series of nucleic acids which are transcribed but not translated. Each polynucleotide generally has a UTR flanking each terminus of the ORF: a 5′ UTR which begins at the transcription start site and continues to the start codon but does not include the start codon, and a 3′ UTR, which begins immediately following the stop codon and continues until the transcriptional termination signal.

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

The term “vaccine,” as used herein, refers to one or more agents administered to a subject in order to stimulate the production of antibodies and provide immunity against one or more diseases.

The term “effective amount” or “therapeutically effective amount,” as used herein, refers to an amount of a biologically active agent (e.g., a vaccine) that is sufficient to elicit a desired biological response. For example, in some embodiments, an effective amount of a vaccine comprising modified RNA encoding RALDH may refer to the amount of vaccine necessary to treat a given disease or disorder, e.g., to generate a therapeutically effective antigen-specific immune response in the subject. As will be appreciated by the skilled artisan, the effective amount of an agent, e.g., a vaccine, may vary depending on various factors as, for example, on the desired biological response, e.g., on the specific antigen used, disease targeted, and on the modified RNA being used.

The terms “administer,” “administering,” or “administration,” as used herein refers to implanting, applying, absorbing, ingesting, injecting, or inhaling, the inventive polynucleotide (e.g., RNA), vaccine, or pharmaceutical composition thereof.

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development.

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

### DETAILED DESCRIPTION OF CERTAIN EMBODIMENTS OF THE INVENTION

The present invention is based, at least in part, on the discovery that modified RNA encoding retinaldehyde dehydrogenase (RALDH) (modRNA-RALDH) can be used in vaccines to target immune response to mucosal tissues. As described herein, the use of modRNA-RALDH in parenteral vaccines leads to mucosal tissue homing, while avoiding exposure to toxic molecules, such as all-trans retinoic acid (ATRA). As a result, vaccines incorporating the modRNA-RALDH described herein are more efficacious than current mucosal tissue vaccines, which often have disagreeable tolerogenic effects. Many current mucosal vaccines require life-attenuated pathogens and/or potent adjuvants, resulting in suboptimal safety profiles. In contrast, the vaccines described herein are able to robustly target mucosal tissues without such side effects. It was also surprisingly found that the mucosal immune surveillance conveyed by ATRA, for example, resulting from administration of the vaccine formulations described herein, was not limited to only the small and large intestine, but extended to other mucosal surfaces, such as the oral cavity, nasal cavity, and urogenital tract. Accordingly, the modRNA-RALDH molecules provided herein may be formulated as pharmaceutical compositions and/or vaccines, and used to treat a number of diseases, including mucosal infections and cancers.

Some aspects of this disclosure provide modified mRNA (modRNA) encoding at least one RALDH enzyme (modRNA-RALDH). Without wishing to be bound by any particular theory, administration of the modRNA-RALDH results in the transient expression of RALDH, and, in the presence of vitamin A, generation of all-trans retinoic acid (ATRA) by antigen-presenting cells (e.g., dendritic cells). Antigen presentation by dendritic cells (DCs) exposed to modRNA-RALDH results in the upregulation of mucosal homing receptors on activated B and T cells. Thus, parenteral vaccine formulations containing modRNA-RALDH can generate antigen-specific immunity at mucosal tissues, without compromising the systemic immune surveillance, unlike other parenteral vaccines.

This mucosal immune surveillance is of relevance in the context of mucosal vaccination strategies targeting a host of mucosal pathogens, including, but not limited to, Shigella, enterotoxigenic E. coli, rotavirus, Chlamydia, and HIV-1. The use of modRNA-RALDH enhances the efficacy of parenterally (i.e., subcutaneously (SQ) or intra-muscularly (IM)) administered vaccines. Parenteral vaccines typically induce immune responses in skin-associated peripheral lymph nodes that drain the inoculation site and normally do not produce mucosa-tropic memory cells. By contrast, oral vaccination can naturally elicit mucosal memory, primarily focused on the small intestine, because APCs in gut-associated lymphoid tissues, unlike APCs in peripheral lymph nodes or the spleen, express RALDH and synthesize all-trans retinoic acid (ATRA). When lymphocytes are activated by antigen and simultaneously exposed to ATRA, they initiate a mucosa-homing program. However, vaccines applied to mucosal surfaces (e.g., orally or intranasally (IN)) are often poorly immunogenic and/or have suboptimal safety profiles due to the need of using either life attenuated pathogens or potent adjuvants that may exert toxic effects. It has been shown that the addition of soluble ATRA to vaccine formulations administered parenterally can induce a protective gut-homing memory response because after SQ injection, free ATRA enters local lymph vessels and is transported together with other vaccine components (e.g., antigen(s) plus adjuvant) to the draining lymph node. However, since free lymph-borne ATRA is not retained in lymph nodes, daily repeat injections over several (e.g., 5) days of relatively high doses of ATRA are needed to achieve mucosal imprinting. This is both impractical for clinical translation and poses a safety risk because high tissue concentrations of free ATRA can cause an inflammatory response at the injection site.

At the intestinal mucosa, dendritic cells (DCs) from gut-associated lymphoid structures (Peyer's patches (PP) and mesenteric lymph nodes (mLN)) induce the up-regulation of α4β7 integrin and chemokine receptor CCR9 on T and B lymphocytes (4, 9-11). As a result, these cells acquire the capacity to home to the small intestine. DCs originating from spleen or peripheral lymph nodes (pLN) cannot induce a similar gut-homing phenotype. This intestinal imprinting has been shown to result from the exclusive capacity of DCs from PP and mLN, but not pLN, to express retinal dehydrogenases (RALDH (9)), enzymes that convert dietary vitamin A to all-trans retinoic acid (ATRA (9)). Of note, the addition of exogenous ATRA to pLN DCs enables these cells to efficiently upregulate gut-homing receptors on activated lymphocytes. Experiments with human lymphocytes and DCs isolated from the mLN vs. spleen yielded analogous results (11). The exposure to ATRA during parenteral immunization was shown to convey protection against Salmonella infections (12). Furthermore, it was found that mucosal immune surveillance conveyed by vaccine formulations containing ATRA is not limited to the small and large intestine, but parenteral ATRA exposure during immunization also generates antigen-specific T and B cells at other mucosal surfaces, such as the oral cavity (determined in the saliva), nasal cavity, and urogenital tract.

Accordingly, vaccines were designed to deliver RALDH enzymes to pLN DCs in order to generate ATRA capable of mucosal imprinting of antigen-specific B and T cells, which would confer a mucosal homing phenotype upon the impacted B and T cells. As demonstrated herein, the use of modRNA-RALDH in parenteral vaccines does allow for mucosal imprinting while avoiding tissue exposure to toxic ATRA; administration of modRNA-RALDH results in the transient expression of RALDH and, in the presence of vitamin A, generation of ATRA by antigen-presenting cells (e.g., DCs). Antigen presentation by DCs exposed to modRNA-RALDH results in the upregulation of mucosal homing receptors on activated B and T cells. Therefore, the vaccine formulations described herein generate antigen-specific immunity at mucosal tissues without compromising systemic immune surveillance, unlike classic parenteral vaccines. modRNA, in free form or packaged in cationic lipid nanoparticles, was found to have moderate adjuvant properties, act directly on cells with phagocytic capacity, and to be degraded intracellularly upon transient induction of protein expression. In addition, modRNA-RALDH can be readily administered either SQ or IM and can be combined with existing or new vaccine formulations to improve efficacy. Moreover, when studying the effect of ATRA on immune responses in skin-draining lymph nodes in vivo, it was found that mucosal memory responses were not only enhanced in the small intestine, but also at other mucosal surfaces, such as the female reproductive tract, the upper respiratory tract, and salivary glands. This finding was unexpected because earlier in vitro studies had suggested that the exposure of activated lymphocytes to ATRA selectively induces homing only to the small intestine, but not to other mucosal tissues. Thus, in addition to preventing intestinal infections, vaccine formulations that generate a sufficient amount of ATRA in peripheral lymph nodes may also have utility in preventing many other mucosal infections.

Hence, the modRNA-RALDH described herein allows for the design of robust parenteral vaccine formulations targeting mucosal tissues, bypassing the tolerogenic effects commonly associated with mucosal vaccinations, as well as toxicity mediated by other molecules with mucosal imprinting properties. Moreover, the induction of mucosal receptors on antigen-specific T cells by modRNA-RALDH means it may also be used in cancer immunotherapy, particularly in regard to tumors developing at mucosal surfaces.

Accordingly, the present invention is directed to modified polynucleotides encoding RALDH protein (modRNA-RALDH), as well as pharmaceutical compositions and vaccines comprising the modRNA-RALDH. The invention also includes methods of using the modRNA-RALDH, for example, to treat various diseases, including viral infections and/or cancers. A further aspect of the invention includes kits including the modRNA-RALDH.

### Modified Polynucleotide Encoding Retinaldehyde Dehydrogenase (RALDH)

Some aspects of this disclosure provide modified polynucleotides (e.g., modRNA) comprising an open reading frame (ORF) encoding a retinaldehyde dehydrogenase (RALDH) protein and functional fragments and variants thereof. In some embodiments, the RALDH is a human RALDH. In other embodiments, the RALDH is from a non-human source (e.g., Mus musculus, Rattus norvegicus, Drosophilia melanogaster, Bifidobacterium bifidum, Micromonas commode, Clostridioides difficile, Ralstonia solanacearum, Flavobacterium psychrophilum, Acinetobacter pittii, Clostridium botulinum, Bordetella bronchiseptica, and Shigella flexneri).

As discussed above, there are three isoforms of RALDH: RALDH1, RALDH2, and RALDH3. Provided below are three exemplary human RALDH ORF polynucleotide sequences:

Aldh1a1
(SEQ ID NO: 1)
AUGUCUUCGCCUGCACAACCUGCAGUCCCUGCCCCACUGGCCGACUUGAAGAUUCAACAU
ACCAAGAUCUUCAUAAACAAUGAAUGGCACAAUUCAGUGAGCGGCAAGAAAUUUCCAGUU
CUUAACCCUGCAACUGAGGAGGUCAUCUGCCACGUGGAAGAAGGGGACAAGGCUGAUGUU
GACAAAGCUGUGAAGGCUGCAAGACAGGCUUUCCAGAUUGGCUCUCCAUGGCGCACCAUG
GAUGCUUCAGAGAGGGGCCGCCUGCUGAACAAGCUGGCUGACUUAAUGGAGAGAGAUCGU
CUGCUGCUGGCUACAAUGGAGGCACUCAAUGGUGGGAAAGUCUUUGCCAAUGCAUACUUG
UCGGAUUUAGGAGGCUGCAUAAAAGCAUUAAAGUACUGUGCAGGCUGGGCUGACAAGAUU
CAUGGUCAAACAAUACCAAGUGAUGGAGACAUUUUCACUUAUACAAGACGUGAACCUAUU
GGAGUGUGUGGCCAAAUCAUCCCCUGGAAUUUUCCAAUGCUCAUGUUCAUUUGGAAGAUA
GGCCCUGCCCUUAGCUGUGGGAAUACCGUGGUUGUCAAGCCAGCAGAGCAAACUCCUCUC
ACGGCUCUUCACCUGGCAUCUUUAAUAAAAGAGGCAGGGUUUCCUCCUGGCGUGGUAAAC
AUUGUCCCUGGUUAUGGGCCAACUGCAGGGGCAGCCAUCUCCUCUCACAUGGAUGUCGAC
AAGGUGGCCUUCACUGGAUCAACACAGGUUGGCAAGUUAAUCAAGGAAGCUGCAGGGAAA
AGCAAUCUGAAGAGAGUCACCCUGGAGCUGGGGGGAAAGAGCCCUUGCAUUGUGUUUGCA
GAUGCCGACUUGGACAUUGCUGUUGAGUUUGCACACCAUGGAGUGUUUUAUCAUCAAGGC
CAAUGUUGUGUCGCAGCAUCCCGGAUUUUUGUUGAGGAGUCAGUUUAUGAUGAGUUUGUG
AAAAGGAGUGUUGAGCGAGCUAAGAAAUAUGUUCUUGGAAAUCCUCUGACCCCAGGAAUA
AAUCAAGGCCCUCAGAUUGACAAGGAACAACAUGAUAAAAUACUCGAUCUCAUUGAGAGU
GGGAAGAAAGAAGGAGCCAAACUGGAGUGUGGUGGAGGACGCUGGGGGAACAAAGGCUUC
UUUGUGCAGCCCACAGUGUUCUCCAACGUGACUGAUGAGAUGCGCAUUGCCAAAGAGGAG
AUAUUUGGACCAGUGCAACAAAUCAUGAAGUUUAAGUCUGUAGAUGAUGUGAUCAAGAGA
GCAAACAAUACUACCUAUGGUUUAGCAGCAGGACUCUUCACUAAAGACCUGGAUAAGGCC
AUCACUGUGUCAUCUGCUCUGCAGGCUGGGGUGGUGUGGGUUAACUGCUAUAUGAUGUUG
UCAGCCCAGUGCCCCUUCGGUGGAUUCAAGAUGUCUGGAAAUGGAAGAGAACUGGGUGAA
CAUGGUCUUUAUGAAUACACUGAGCUCAAGACAGUCGCAAUGAAGAUAUCUCAGAAGAAC
UCCUAA
Aldh1a2
(SEQ ID NO: 2)
AUGACUUCCAGCGAGAUCGCCAUGCCGGGCGAGGUGAAGGCCGACCCCGCCGCGCUCAUG
GCCUCGCUGCAGCUCCUGCCGUCGCCCACGCCCAACCUCGAGAUCAAGUACACCAAGAUC
UUUAUUAACAAUGAAUGGCAGAACUCAGAGAGUGGGAGAGUGUUCCCUGUCUGUAAUCCA
GCCACAGGAGAGCAAGUGUGUGAAGUUCAAGAAGCAGACAAGGUGGAUAUAGACAAGGCA
GUGCAAGCAGCCCGCCUUGCCUUCUCUCUUGGUUCUGUGUGGAGAAGGAUGGAUGCGUCU
GAAAGAGGACGUCUGUUGGACAAGCUUGCAGACUUGGUGGAACGGGACAGGGCAACUCUU
GCAACUAUGGAAUCCCUAAAUGGCGGUAAGCCAUUCCUGCAAGCUUUUUACAUCGAUUUG
CAGGGAGUCAUCAAAACCCUGAGGUAUUAUGCAGGCUGGGCUGAUAAAAUUCACGGAAUG
ACCAUUCCUGUAGAUGGAGACUAUUUUACUUUCACAAGACACGAGCCCAUUGGAGUGUGU
GGACAGAUCAUCCCGUGGAACUUCCCCCUGCUGAUGUUCACCUGGAAAAUUGCUCCCGCA
UUGUGCUGUGGUAACACCGUGGUCAUCAAACCCGCUGAGCAGACACCGCUCAGUGCACUG
UAUAUGGGAGCCCUCAUCAAGGAGGCUGGCUUUCCACCCGGAGUCGUCAAUAUCCUGCCA
GGGUAUGGGCCAACAGCAGGGGCAGCAAUCGCUUCUCACAUCGGCAUAGACAAGAUUGCA
UUCACAGGCUCUACUGAGGUUGGAAAGCUUAUUCAAGAAGCAGCUGGAAGAAGUAACCUG
AAGAGAGUGACCCUGGAACUCGGAGGGAAGAGCCCUAAUAUUAUUUUUGCAGAUGCUGAC
UUGGACUACGCUGUGGAGCAGGCUCACCAGGGUGUGUUCUUCAACCAAGGCCAGUGCUGC
ACCGCAGGGUCUCGCAUCUUUGUGGAAGAAUCCAUCUAUGAGGAAUUUGUGAAAAGAAGU
GUGGAAAGGGCCAAGAGGCGCAUUGUGGGGAGCCCCUUUGAUCCCACAACUGAGCAGGGU
CCUCAGAUCGAUAAGAAACAAUACAACAAAGUCCUGGAGCUUAUCCAGAGCGGUGUGGCU
GAGGGCGCCAAGCUGGAGUGCGGAGGCAAAGGGCUGGGAAGGAAGGGCUUCUUCAUUGAG
CCCACCGUGUUCUCCAACGUCACUGAUGACAUGCGGAUUGCCAAGGAGGAGAUCUUUGGU
CCUGUUCAAGAAAUUUUGAGGUUUAAGACUAUGGAUGAAGUUAUAGAAAGAGCCAAUAAC
UCAGACUUUGGACUUGUAGCAGCUGUCUUCACUAAUGACAUCAACAAGGCUCUCAUGGUA
UCCUCCGCAAUGCAAGCUGGGACAGUUUGGAUCAAUUGUUACAAUGCCUUAAAUGCGCAG
AGCCCCUUUGGGGGGUUCAAGAUGUCUGGAAAUGGGAGAGAAAUGGGUGAGUUUGGCUUA
CGGGAGUAUUCAGAAGUAAAGACCGUGACGGUGAAGAUCCCCCAGAAGAACUCCUAA
Aldh1a3
(SEQ ID NO: 3)
AUGGCUACCACCAACGGGGCUGUGGAAAACGGACAGCCGGAUGGGAAACCGCCUGCCUUG
CCGCGCCCCAUCCGCAACUUGGAGGUCAAGUUCACCAAGAUAUUUAUCAACAACGACUGG
CACGAAUCCAAGAGUGGAAGAAAGUUUGCCACAUAUAACCCUUCAACACUAGAGAAAAUA
UGUGAGGUGGAAGAAGGAGAUAAGCCCGAUGUGGACAAGGCUGUGGAGGCCGCUCAAGCU
GCCUUCCAGCGGGGAUCCCCGUGGCGCCGGCUGGAUGCACUGAGCAGAGGCCAGUUGCUG
CAUCAGCUGGCUGACCUUGUAGAAAGGGACCGAGCGAUCCUGGCUACUCUGGAGACCAUG
GACACCGGCAAGCCAUUCCUUCAUGCCUUUUUCGUCGACCUGGAAGGCUGUAUUAAGACC
UUCAGAUAUUUUGCCGGGUGGGCAGACAAAAUCCAGGGCAGGACCAUCCCCACAGAUGAC
AACGUUGUGUGCUUCACCAGGCAUGAGCCCAUCGGGGUGUGUGGGGCCAUUACACCAUGG
AACUUCCCCCUGCUGAUGCUGGCCUGGAAACUGGCUCCUGCCCUGUGCUGUGGGAACACC
GUGGUCCUGAAGCCAGCUGAGCAGACCCCUCUCACGGCUCUGUACCUAGCCUCUCUCAUC
AAAGAGGUCGGGUUCCCUCCGGGUGUGGUGAACAUUGUACCAGGCUUUGGGCCCACUGUG
GGAGCAGCAAUUUCCUCCCAUCCGCAGAUCAACAAGAUAGCCUUCACCGGCUCCACAGAG
GUUGGAAAGCUGGUCAGAGAAGCCGCCUCCCGGAGCAACCUGAAGAGGGUCACACUGGAG
CUAGGAGGCAAGAACCCGUGCAUCGUGUGUGCAGAUGCUGACUUGGACUUGGCCGUCGAG
UGUGCUCACCAGGGAGUGUUCUUCAACCAAGGCCAGUGCUGUACAGCGGCCUCCAGGGUG
UUCGUGGAAGAGCAGGUCUACGGGGAGUUUGUGAGGAGGAGUGUGGAGUUCGCCAAGAAG
AGGCCAGUUGGAGACCCCUUCGAUGCCAAAACGGAGCAGGGGCCUCAGAUCGACCAAAAG
CAGUUUGACAAAAUCCUCGAGCUGAUUGAGAGUGGGAAGAAGGAAGGGGCCAAGCUAGAA
UGUGGGGGGUCAGCCAUGGAGGACAGAGGGCUGUUCAUCAAACCCACGGUCUUCUCAGAU
GUUACGGACAACAUGAGGAUUGCCAAAGAGGAGAUUUUCGGACCAGUGCAGCCGAUCCUG
AAGUUCAAAAACCUGGAGGAGGUGAUCAAGAGAGCGAAUAGCACCGACUAUGGACUCACA
GCAGCAGUGUUCACCAAAAACCUGGACAAAGCACUGAAGCUGGCUGCUGCGCUCGAGUCG
GGGACAGUCUGGAUCAACUGCUACAAUGCAUUUUAUGCACAGGCUCCAUUUGGUGGCUUC
AAAAUGUCUGGGAAUGGCAGAGAACUAGGAGAAUAUGCUCUGGCUGAAUAUACAGAAGUG
AAAACUGUCACCAUCAAACUCGAGGAGAAGAACCCCUGA

In some embodiments, the ORF of the modRNA comprises a sequence 100% identical to any one of SEQ ID NOs: 1-3. In another embodiment, the ORF of the modRNA comprises a sequence that is 65%, 70%, 75%, 80%, 85%, 90%, 91%, 92%, 93%, 94%, 95%, 96%, 97%, 98%, 99%, or more identical to any one of SEQ ID NOs: 1-3.

In some embodiments, the ORF of the modRNA may encode RALDH1, RALDH2, or RALDH3. In one embodiment, the ORF of the modRNA encodes RALDH1. In one embodiment, the ORF of the modRNA encodes RALDH2. In one embodiment, the ORF of the modRNA encodes RALDH3. In another embodiment, the ORF of the modRNA encodes two of the following: RALDH1, RALDH2, and RALDH3. In other embodiments, the modRNA encodes RALDH1, RALDH2, and RALDH3.

Exemplary sequences of human RALDH1, RALDH2, and RALDH3 are presented herein as SEQ ID NOs: 4-6. In some embodiments, the ORF encodes a RALDH protein that is 100% identical to any one of SEQ ID NOs: 4-6. In other embodiments, the ORF encodes a RALDH protein that is 65%, 70%, 75%, 80%, 85%, 90%, 91%, 92%, 93%, 94%, 95%, 96%, 97%, 98%, 99%, or more identical to any one of SEQ ID NOs: 4-6.

RALDH typically comprises three domains: a NAD⁺-binding domain (comprising a five-stranded parallel β-sheet), a catalytic domain (comprising a six-stranded parallel β-sheet), and an oligomerization domain (comprising a three-stranded anti-parallel β-sheet). In some embodiments, the ORF of the modRNA may encode one or more domains of RALDH1, RALDH2, and RALDH3, or a combination thereof. For example, the ORF may comprise a first domain from RALDH1, a second domain from RALDH2, and a third domain from RALDH3. In some embodiments, the ORF encodes two domains from a first RALDH protein and a single domain from a second RALDH protein.

In some embodiments, the modRNA is a nucleic acid molecule that has undergone a molecular biological manipulation, i.e., non-naturally occurring nucleic acid molecule or genetically engineered nucleic acid molecule. Furthermore, “modRNA” refers to a nucleic acid sequence which is not naturally occurring, or can be made by the artificial combination of two otherwise separated segments of nucleic acid sequence, i.e., by ligating together pieces of DNA that are not normally continuous. The artificial combination may be accomplished by either chemical synthesis means, or by the artificial manipulation of isolated segments of nucleic acids, e.g., by genetic engineering techniques using restriction enzymes, ligases, and similar recombinant techniques as described by, for example, Sambrook et al., Molecular Cloning, second edition, Cold Spring Harbor Laboratory, Plainview, N.Y.; (1989), or Ausubel et al., Current Protocols in Molecular Biology, Current Protocols (1989), and DNA Cloning: A Practical Approach, Volumes I and II (ed. D. N. Glover) IREL Press, Oxford, (1985); each of which is incorporated herein by reference.

Such manipulation may be done to replace a codon with a redundant codon encoding the same or a conservative amino acid, while typically introducing or removing a sequence recognition site. Alternatively, it may be performed to join together nucleic acid segments of encoding different RALDH proteins or domains to generate a single genetic entity comprising a desired combination of RALDH not found in nature. Restriction enzyme recognition sites are often the target of such artificial manipulations, but other site specific targets, e.g., promoters, DNA replication sites, regulation sequences, control sequences, open reading frames, or other useful features may be incorporated by design.

The modRNA may be modified in a number of ways. In some embodiments, the modRNA comprises natural nucleosides (e.g. adenosine, thymidine, guanosine, cytidine, uridine, deoxyadenosine, deoxythymidine, deoxyguanosine, and deoxycytidine) in addition to one or more of: nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); isomers (e.g., pseudouridine), and/or modified phosphate groups (e.g., phosphorothioates and 5′-N-phosphoramidite linkages). Other exemplary modifications include, but are not limited to, methyladenosine, 6-methyladenosine, 5-hydroxymethylcytidine, 5-formylcytidine, 2-thiouridine, and inosine. In some embodiments, the modRNA comprises at least one uridine that is pseudouridine and at least one cytosine that is 5-methylcytosine. Such modifications may increase transcription, reduce or eliminate immunogenicity, and/or reduce degradation of the polynucleotide, among other properties, compared to an unmodified polynucleotide.

In some embodiments, all of the uridine bases of the modRNA are pseudouridine. In other embodiments, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more of the uridine bases of a modRNA are pseudouridine.

In some embodiments, all of the cytosine bases of the modRNA are 5-methylcytosine. In other embodiments, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more of the cytosine bases of a modRNA are 5-methylcytosine.

In some embodiments, the modRNA further comprises a 5′ untranslated region (UTR) and a 3′ UTR. UTRs have regulatory functions; for example, the 5′ UTR typically has a role in translation initiation and may include a Kozak sequence. The consensus Kozak sequence, CCR(A/G)CCAUGG (where R is a purine (adenine or guanine) three bases upstream of a start codon (AUG), followed by another guanine), has been shown to be involved in ribosomal initiation of the translation of many genes. In some embodiments, the polynucleotide is enzymatically capped, for example, with a 5′ terminal cap, such as 7 mG(5)ppp(5′)NlmpNp. Likewise, the 3′ UTR may comprise a number of adenosines and uridines. In some embodiments, the 3′ UTR comprises a poly(A) sequence. In certain embodiments, the poly(A) tail enhances the expression level of the encoded protein.

The 5′ UTR and/or the 3′ UTR may be heterologous or synthetic. In some embodiments, the 5′ UTR may be heterologous, and the 3′ UTR may be synthetic. In another embodiment, the 5′ UTR may be synthetic, and the 3′ UTR may be heterologous. Any UTR from any gene may be engineered into the modified polynucleotide (e.g., RNA) described herein.

In some embodiments, for example, when the modRNA encodes more than one RALDH protein, the modRNA further comprises one or more ribosome-binding sites (RBS). A RBS is a sequence of nucleotides upstream of the start codon of a mRNA transcript that recruits a ribosome during initiation of protein translation. In other embodiments, Internal Ribosome Entry Sites (IRESs) and/or one or more 2A peptides. IRESs are elements that permit initiation of translation from an internal region of an mRNA, whereas 2A peptides cause the ribosome to skip the synthesis of a peptide bond located in the C-terminus of the 2A element, resulting in a separation between the end of the 2A sequence and the next peptide downstream. Examples of 2A peptides include, but are not limited to, T2A, P2A, E2A, and F2A.

The modRNA may comprise further elements, such as natural regulatory (expression control) sequences or may be associated with heterologous sequences, including, promoters, enhancers, response elements, suppressors, signal sequences, and introns.

In some embodiments, the modRNA may be codon-optimized (e.g. the codon AGT being used instead of AGC for coding of the amino acid serine; TTT/TTC for phenylalanine; TTA/TTG/CTT/CTC/CTA/CTG for leucine; ATT/ATC/ATA for isoleucine; GTT/GTC/GTA/GTG for valine; TCT/TCC/TCA/TCG for serine; CCT/CCC/CCA/CCG for proline/ACT/ACC/ACA/ACG for threonine; GCT/GCC/GCA/GCG for alanine; TAT/TAC for tyrosine; CAT/CAC for histidine; CAA/CAG for glutamine; AAT/AAC for asparagine; AAA/AAG for lysine; GAT/GAC for aspartic acid; GAA/GAG for glutamic acid; TGT/TGC for cysteine; CGT/CGC/CGA/CGG/AGA/AGG for arginine; GGT/GGC/GGA/GGG for glycine). The optimized codons can be used, for example, to increase mRNA stability, reduce secondary structures, minimize tandem repeat codons or base runs (which may impair gene construction or expression), manipulate transcriptional and translational control regions, add or delete protein trafficking sequences, insert, delete, or shuffle protein domains, add or delete restriction sites, or match codon frequencies in target and host organisms (for proper folding and secondary structure). Codon optimization tools, algorithms and services are known in the art, and non-limiting examples include services from GeneArt (Life Technologies), DNA2.0 (Menlo Park CA) and/or proprietary methods.

The modRNA described herein may be synthesized by standard methods known in the art, e.g., by in vitro or in vivo transcription. DNA templates may be incorporated into a wide variety of vectors that incorporate suitable RNA polymerase promoters such as the T7 or SP6 polymerase promoters, resulting in production of the desired RNA. Alternatively, cDNA constructs that synthesize RNA constitutively or inducibly, depending on the promoter used, can be introduced stably into cell lines, resulting in the production of the desired RNA.

### Vaccines

In one aspect of the disclosure, the modRNA is provided as a component of a vaccine. The vaccine, in some embodiments, comprises at least one antigen and at least one RNA polynucleotide (e.g., mRNA) comprising an ORF encoding a RALDH protein. The vaccine, in a further embodiment, may include an adjuvant and/or a pharmaceutically acceptable excipient. In some embodiments, the vaccine is formulated in a nanoparticle, microparticle, hydrogel, or liposome. In some embodiments, the vaccine formulation includes at least one substrate (direct or indirect) of RALDH.

In one embodiment, the vaccine comprises at least one first RNA polynucleotide (e.g., mRNA) comprising an ORF encoding at least one antigenic polypeptide or immunogenic polypeptide fragment from one or more pathogens and at least one second RNA polynucleotide (e.g., mRNA) comprising an ORF encoding a RALDH protein. In some embodiments, the second RNA polynucleotide comprises an ORF wherein at least one uridine is pseudouridine and/or at least one cytosine is 5-methylcytosine.

In some embodiments, the vaccine comprises at least one RNA polynucleotide (e.g., mRNA) comprising an ORF encoding a RALDH protein. For example, the vaccine may comprise 1, 2, 3, 4, 5, 6, 7, 8, 9, 10, or more RNA polynucleotides (e.g., mRNAs) encoding one or more RALDH proteins. In one embodiment, the vaccine comprises at least one RNA polynucleotide comprising an ORF encoding a RALDH1 protein. In one embodiment, the vaccine comprises at least one RNA polynucleotide comprising an ORF encoding a RALDH2 protein. In one embodiment, the vaccine comprises at least one RNA polynucleotide comprising an ORF encoding a RALDH3 protein. In some embodiments, the vaccine comprises an RNA polynucleotide comprising an ORF encoding a RALDH1 protein, and an RNA polynucleotide comprising an ORF encoding a RALDH2 protein. In other embodiments, the vaccine comprises at least one RNA polynucleotide comprising an ORF encoding a RALDH1 protein, and at least one RNA polynucleotide comprising an ORF encoding a RALDH3 protein. In some embodiments, the vaccine comprises at least one RNA polynucleotide comprising an ORF encoding a RALDH2 protein, and at least one RNA polynucleotide comprising an ORF encoding a RALDH3 protein. In some embodiments, the vaccine comprises an RNA polynucleotide comprising an ORF encoding a RALDH1 protein, an RNA polynucleotide comprising an ORF encoding a RALDH2 protein, and an RNA polynucleotide comprising an ORF encoding a RALDH3 protein.

In some embodiments, the ORF of the modRNA may encode one or more domains of RALDH1, RALDH2, and RALDH3, or a combination thereof. For example, the ORF may comprise a first domain from RALDH1, a second domain from RALDH2, and a third domain from RALDH3. In some embodiments, the ORF encodes two domains from a first RALDH protein and a single domain from a second RALDH protein. In a further embodiment, the ORF of the modRNA may encode one or more truncated RALDH proteins. For example, the ORF of the modRNA may encode a truncated RALDH1 protein, a truncated RALDH2 protein, a truncated RALDH3 protein, or any combination thereof.

In some embodiments, the vaccine further comprises an adjuvant. Adjuvants may be used, for example, to enhance an immune response and/or to improve the efficacy of a vaccine. Adjuvants include, but are not limited to, monophosphoryl lipid A (MPL), MF59 (squalene), adjuvant system (AS) 01, AS02, AS03, AS04, alum, CAF01, IC31® (Valneva Technologies), iscomatrix, and TLR agonists. Examples of TLR agonists include Pam3Cys, BCG, LPS, 852A, VTX-2337, Poly(I:C), imidazoquinolines, and CpG. In one embodiment, the adjuvant is alum. In another embodiment, the adjuvant is one or more TLR agonists.

In further embodiments, the vaccine further comprises one or more components of the retinoic acid synthesis pathway. Examples of such components include, but are not limited to, retinal, β-carotenes, and vitamin A. In some embodiments, the vaccine comprises a combination of such components. In other embodiments, the vaccine comprises one of the components.

In one embodiment, the vaccine is a mucosal vaccine. The mucosal vaccine may be targeted to different mucosal tissues, including, but not limited to, the oral cavity, the nasal cavity, the eye, the respiratory tract, the intestine, and the urogenital tract.

In some embodiments, the vaccine is monovalent. In other embodiments, the vaccine is multivalent, and is capable of protecting against more than one strain of the same microorganism and/or more than one microorganism. Such vaccines may include 2, 3, 4, 5, 6, 7, 8, 9, 10, or more different antigenic polypeptides/polynucleotides and/or immunogenic polypeptide/polynucleotide fragments from one or more pathogens (e.g., mucosal pathogens).

In some embodiments, the at least one antigen may comprise a polynucleotide, protein, peptide, plasmid, virus, bacteria, bacterial fragment, fungus, fungal fragment and/or a conjugate. Other antigens are known in the art, and are contemplated herein.

The pathogens (e.g., mucosal pathogens), in some embodiments, are viral or bacterial pathogens, or a combination thereof. Exemplary bacterial pathogens include a Bacillus species, a Bartonella species, a Bordetella species, a Borrelia species, a Campylobacter species, a Chlamydia species, a Chlamydophila species, a Clostridium species, a Corynebacterium species, an Enterococcus species, an Escherichia species, a Francisella species, a Haemophilus species, a Helicobacter species, a Legionella species, a Leptospira species, a Listeria species, a Mycobacterium species, a Mycoplasma species, a Neisseria species, a Pseudomonas species, a Rickettsia species, a Salmonella species, a Shigella species, a Staphylococcus species, a Streptococcus species, a Treponema species, an Ureaplasma species, a Vibrio species, and a Yersinia species. In one particular embodiment, the bacterial pathogen is a Chlamydia species, for example, Chlamydia trachomatis.

Exemplary viral pathogens include, but are not limited to, the following: Aichi virus, Astrovirus, Australian bat lyssavirus, Banna virus, Barmah forest virus, Bunyamwera virus, Bunyavirus, Cercopithecine herpesvirus, Chandipura virus, Chikungunya virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, Hantaan virus, Hendra virus, Hepatitis A virus, Hepatitis B virus, Hepatitis C virus, Hepatitis E virus, Hepatitis delta virus, Human adenovirus, Human astrovirus, Human coronavirus, Human cytomegalovirus, Human enterovirus 68, 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16,18, Human parainfluenza, Human parvovirus B19, Human respiratory syncytial virus, Human rhinovirus, Human T-lymphotropic virus, Human torovirus, Influenza A virus, Influenza B virus, Influenza C virus, JC polyomavirus, Japanese encephalitis virus, Junin arenavirus, KI Polyomavirus, Kunjin virus, Lagos bat virus, Lake Victoria Marburgvirus, Langat virus, Lassa virus, Lordsdale virus, Louping ill virus, Lymphocytic choriomeningitis virus, Machupo virus, Mastadenovirus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norovirus (Norwalk virus), O'nyong-nyong virus, Orf virus, Oropouche virus, Pichinde virus, Poliovirus, Punta toro phlebovirus, Puumala virus, Rabies virus, Rift valley fever virus, Ross river virus, Rotavirus A, Rotavirus B, Rotavirus C, Rubella virus, Sagiyama virus, Salivirus A, Sandfly fever Sicilian virus, Sapporo virus, Semliki forest virus, Seoul virus, Sindbis virus, Southampton virus, St. Louis encephalitis virus, Tick-borne powassan virus, Toscana virus, Uukuniemi virus, Varicella-zoster virus, Venezuelan equine encephalitis virus, Vesicular stomatitis virus, Western equine encephalitis virus, West Nile virus, Yellow fever virus, and Zika virus.

In some embodiments, the mucosal vaccine includes a recombinant or purified antigen or a mixture of antigens from one or more mucosal pathogens. Examples of antigens from mucosal pathogens include, but are not limited to: capsular polysaccharides (e.g., Streptococcus species), chlamydia protease-like activity factor (CPAF; e.g., Chlamydia species), Cwp66 and Cwp84 (Clostridium species), fimbrial adhesins (e.g., Escherichia species), flagellin (e.g., Escherichia species, Salmonella species, Campylobacter species), glycan polymers contained within a bacterial lipopolysaccharide (e.g., O-antigen; Escherichia species, Salmonella species, Shigella species), glycoconjugates (e.g., Escherichia species, Salmonella species, Shigella species, Streptococcus species), heat-labile toxin subunits (LT; e.g., Escherichia species), glycoprotein VP7 (Rotavirus), hemagglutinin antigens (e.g., Influenza A virus), major outer membrane protein (MOMP; e.g., Chlamydia species), matrix proteins (e.g., Influenza A virus), neuroaminidase (e.g., influenza A virus), outer membrane proteins (OMP; e.g., Chlamydia species, Treponema species), periplasmic protein CjaA (Campylobacter species), plasmid glycoprotein 3 (Pgp3; e.g., Chlamydia species), polymorphic membrane proteins (Pmps; e.g., Chlamydia species), pneumococcal surface protein A (PspA; Streptococcus species), pneumolysin (Streptococcus species), S-layer proteins (SLP; e.g., Clostridium species), spike protein VP4 (Rotavirus), spike protein VP8 (Rotavirus), virus-like particles (VLP; e.g., Norwalk virus, Influenza virus).

In some embodiments, the mucosal vaccine includes one or more species of inactivated bacteria, one or more species of live attenuated bacteria, or one or more strains of attenuated or inactivated viruses.

The vaccine, in some embodiments, is formulated in a hydrogel.

The vaccine, in some embodiments, is formulated as a particle (e.g., nanoparticle, microparticle). In some embodiments, the modRNA-RALDH is formulated in the nanoparticle. In one embodiment, the antigen (e.g., the polynucleotide encoding at least one antigenic polypeptide or immunogenic polypeptide fragment from one or more mucosal pathogens) is formulated in the nanoparticle. In another embodiment, an adjuvant is formulated in the nanoparticle. In some embodiments, all three components are formulated in the same nanoparticle. In other embodiments, all three components are formulated in separate nanoparticles. In a further embodiment, two of the components are formulated in a nanoparticle and the third component is either formulated in a separate nanoparticle or not formulated in a nanoparticle. In one specific embodiment, the modRNA-RALDH and the antigen (e.g., the polynucleotide encoding at least one antigenic polypeptide or immunogenic polypeptide fragment from one or more pathogens) are formulated in the same nanoparticle, and an adjuvant is not formulated in a nanoparticle.

In some embodiments, the vaccine formulated in a nanoparticle is on the surface of the nanoparticle (e.g., covalently or non-covalently associated), encapsulated within the nanoparticle (e.g., covalently or non-covalently associated), or both on the surface of the nanoparticle (e.g., covalently or non-covalently associated) and encapsulated within the nanoparticle (e.g., covalently or non-covalently associated). If two or more of the vaccine components are formulated in an nanoparticle, then each component separately may be present on the surface of the nanoparticle (e.g., covalently or non-covalently associated), encapsulated within the nanoparticle (e.g., covalently or non-covalently associated), or both on the surface of the nanoparticle (e.g., covalently or non-covalently associated) and encapsulated within the nanoparticle (e.g., covalently or non-covalently associated).

The nanoparticle, in some embodiments, can be composed of polymer and/or non-polymer molecules. Accordingly, the nanoparticle can be protein-based. The nanoparticle, in some embodiments, is macromolecular. In some embodiments, the nanoparticle is composed of amino acids. A nanoparticle can be, but is not limited to, one or a plurality of lipid-based nanoparticles, polymeric nanoparticles, metallic nanoparticles, surfactant-based emulsions, dendrimers, and/or nanoparticles that are developed using a combination of nanomaterials, such as lipid-polymer nanoparticles.

In some embodiments, the nanoparticle is composed of one or more polymers. Examples of polymers include, but are not limited to, polysaccharides (e.g., alginate, dextran, chitosan, agarose, and pullulan), polypeptides (e.g., albumin, gelatin, lectin, legumine, and viciline), polyesters, polyethers, and polyamides. In some embodiments, the one or more polymers is a water soluble, non-adhesive polymer. In some embodiments, polymer is polyethylene glycol (PEG) or polyethylene oxide (PEO). In some embodiments, the polymer is polyalkylene glycol or polyalkylene oxide. In some embodiments, the one or more polymers is a biodegradable polymer. In some embodiments, the one or more polymers is a biocompatible polymer that is a conjugate of a water soluble, non-adhesive polymer and a biodegradable polymer. In some embodiments, the biodegradable polymer is polylactic acid (PLA), poly(glycolic acid) (PGA), or poly(lactic acid/glycolic acid) (PLGA). In some embodiments, the nanoparticle is composed of PEG-PLGA polymers. In some embodiments, the nanoparticle comprises one or more cationic lipids.

In some embodiments, polymers can be cationic polymers. In general, cationic polymers are able to condense and/or protect negatively charged strands of nucleic acids (e.g., DNA, RNA, or derivatives thereof). Amine-containing polymers such as poly(lysine) (Zauner et al., 1998, Adv. Drug Del. Rev., 30:97; and Kabanov et al., 1995, Bioconjugate Chem., 6:7; both of which are incorporated herein by reference), poly(ethylene imine) (PEI; Boussif et al., 1995, Proc. Natl. Acad. Sci., USA, 1995, 92:7297; incorporated herein by reference), and poly(amidoamine) dendrimers (Kukowska-Latallo et al., 1996, Proc. Natl. Acad. Sci., USA, 93:4897; Tang et al., 1996, Bioconjugate Chem., 7:703; and Haensler et al., 1993, Bioconjugate Chem., 4:372; all of which are incorporated herein by reference) are positively-charged at physiological pH, form ion pairs with nucleic acids, and mediate transfection in a variety of cell lines.

In some embodiments, the nanoparticle is a cationic lipid nanoparticle, such as those described in WO 2015/164674. Non-limiting examples of lipid nanoparticle compositions and methods of making them are described, for example in Semple et al. (2010) Nat. Biotechnol. 28:172-176; Jayarama et al. (2012), Angew. Chem. Int. Ed., 51:8529-8533; and Maier et al. (2013) Molecular Therapy 21, 1570-1578.

In some embodiments, the nanoparticle is formed by self-assembly. Self-assembly refers to the process of the formation of a nanoparticle using components that will orient themselves in a predictable manner forming nanoparticles predictably and reproducibly. In some embodiments, the nanoparticles are formed using amphiphilic biomaterials which orient themselves with respect to one another to form nanoparticles of predictable dimension, constituents, and placement of constituents. According to the invention, the amphiphilic biomaterials may have attached to them at least one of the vaccine components such that when the nanoparticles self assemble, there is a reproducible pattern of localization and density of the agents on/in the nanoparticle.

In some embodiments, the nanoparticle has a positive zeta potential. In some embodiments, the nanoparticle has a net positive charge at neutral pH. In some embodiments, the nanoparticle comprises one or more amine moieties at its surface. In some embodiments, the amine moiety is a primary, secondary, tertiary, or quaternary amine. In some embodiments, the amine moiety is an aliphatic amine. In some embodiments, the nanoparticle comprises an amine-containing polymer. In some embodiments, the nanoparticle comprises an amine-containing lipid. In some embodiments, the nanoparticle comprises a protein or a peptide that is positively-charged at neutral pH. In some embodiments, the nanoparticle is a latex particle. In some embodiments, the nanoparticle with the one or more amine moieties on its surface has a net positive charge at neutral pH.

The nanoparticles of the compositions provided herein, in some embodiments, have a mean geometric diameter that is less than 500 nm. In some embodiments, the nanoparticles have mean geometric diameter that is greater than 50 nm but less than 500 nm. In some embodiments, the mean geometric diameter of a population of nanoparticles is about 60 nm, 75 nm, 100 nm, 125 nm, 150 nm, 175 nm, 200 nm, 225 nm, 250 nm, 275 nm, 300 nm, 325 nm, 350 nm, 375 nm, 400 nm, 425 nm, 450 nm, or 475 nm. In some embodiments, the mean geometric diameter is between 100-400 nm, 100-300 nm, 100-250 nm, or 100-200 nm. In some embodiments, the mean geometric diameter is between 60-400 nm, 60-350 nm, 60-300 nm, 60-250 nm, or 60-200 nm. In some embodiments, the mean geometric diameter is between 75-250 nm. In some embodiments, 30%, 40%, 50%, 60%, 70%, 80%, 90%, or more of the nanoparticles of a population of nanoparticles have a diameter that is less than 500 nM. In some embodiments, 10%, 20%, 30%, 40%, 50%, 60%, 70%, 80%, 90%, or more of the nanoparticles of a population of nanoparticles have a diameter that is greater than 50 nm but less than 500 nm. In some embodiments, 10%, 20%, 30%, 40%, 50%, 60%, 70%, 80%, 90%, or more of the nanoparticles of a population of nanoparticles have a diameter of about 60 nm, 75 nm, 100 nm, 125 nm, 150 nm, 175 nm, 200 nm, 225 nm, 250 nm, 275 nm, 300 nm, 325 nm, 350 nm, 375 nm, 400 nm, 425 nm, 450 nm, or 475 nm. In some embodiments, 10%, 20%, 30%, 40%, 50%, 60%, 70%, 80%, 90%, or more of the nanoparticles of a population of nanoparticles have a diameter that is between 100-400 nm, 100-300 nm, 100-250 nm, or 100-200 nm. In some embodiments, 10%, 20%, 30%, 40%, 50%, 60%, 70%, 80%, 90%, or more of the nanoparticles of a population of nanoparticles have a diameter that is between 60-400 nm, 60-350 nm, 60-300 nm, 60-250 nm, or 60-200 nm.

In some embodiments, the vaccine is a tolerogenic vaccine. In such embodiments, the vaccine comprises a modRNA-RALDH, an immunomodulatory agent, and at least one antigen (e.g., from one or more mucosal pathogens). Immunomodulatory agents, as described herein, are modRNAs capable of reducing an immune response (e.g., promoting a tolerogenic immune response), such as a T cell and/or B cell response. For example, such agents may reduce the number or proliferation of T and/or B cells, and/or may increase the number of T regulatory cells and/or B regulatory cells. Exemplary immunomodulatory agents include, but are not limited to, GM-CSF, TNF-α, mTOR inhibitors, HDAC inhibitors, MHC-peptide complexes, and antigen-laden erythrocytes.

In another embodiment, the vaccine is a cancer vaccine. In such embodiments, the vaccine comprises a modRNA-RALDH, at least one tumor antigen, and at least one adjuvant. For example, the tumor antigen may be a polynucleotide encoding at least one tumor antigen (e.g., mucosal antigen) or immunogenic polypeptide fragment thereof. Exemplary mucosal tumor antigens include, but are not limited to, the following: guanylyl cyclase C, sucrose isomaltase, CDX1, CDX2, mammoglobulin, small breast epithelial mucin, RAGE antigens, and MUC1. In some embodiments, the tumor antigen is one or more neoantigens (e.g., antigens encoded by tumor-specific mutated genes).

The cancer vaccine described herein may further comprise administering to the subject an inhibitor of a checkpoint molecule, an activator of a co-stimulatory receptor, or an inhibitor of an innate immune cell target. Examples of checkpoint molecules include, but are not limited to, PD-1, PD-L1, PD-L2, CTLA-4, LAG3, TIM-3, and A2aR. Examples of co-stimulatory receptors include, but are not limited to, OX40, GITR, CD137, CD40, CD27, and ICOS. Examples of innate immune cell targets include, but are not limited to, KIR, NKG2A, CD96, TLR, and IDO.

In another embodiment, the cancer vaccine is administered in addition to a cell therapy, such as antigen-pulsed antigen-presenting cells (e.g., antigen-pulsed dendritic cells or antigen-loaded T cells or B cells, or PBMC), gene therapy (e.g., SOZ vectors), or with CAR T cell therapy or CAR NK cell therapy.

### Pharmaceutical Compositions

The present disclosure provides pharmaceutical compositions comprising at least one modRNA-RALDH molecule, at least antigen (e.g., the polynucleotide encoding at least one antigenic polypeptide or immunogenic polypeptide fragment from one or more pathogens), and optionally, a pharmaceutically acceptable excipient. In certain embodiments, the vaccine is administered in an effective amount, e.g., a therapeutically effective amount or prophylactically effective amount.

A pharmaceutical composition of the present disclosure can be administered via one or more routes of administration using one or more of a variety of methods known in the art. As will be appreciated by the skilled artisan, the route and/or mode of administration will vary depending on the desired results. Preferred routes of administration include intravenous, intramuscular, intradermal, intraperitoneal, subcutaneous, spinal, or other parenteral routes of administration, for example by epidermal administration (e.g., by injection or infusion). The phrase “parenteral administration,” as used herein, means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. In certain embodiments, the vaccine is administered subcutaneously or intramuscularly. In certain embodiments, the vaccine is administered orally.

Depending on the route of administration, the pharmaceutical composition or vaccine may be coated in a material to protect the modRNA from the action of acids and other natural conditions that may inactivate the modRNA (e.g., gastric acid). In one embodiment, the vaccine is formulated within a nanoparticle, as described above.

Pharmaceutically acceptable excipients include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in the formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21^{st} Edition (Lippincott Williams & Wilkins, 2005).

Pharmaceutical compositions described herein can be prepared by any method known in the art of pharmacology. In general, such preparatory methods include the steps of bringing the active ingredient into association with the excipient and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the vaccine, the pharmaceutically acceptable carrier, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

In some embodiments, the vaccine or pharmaceutical composition may comprise a buffering agent. Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Liquid dosage forms for parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the invention are mixed with solubilizing agents such as Cremophor, alcohols, oils, modified oils, glycols, polysorbates, cyclodextrins, polymers, and combinations thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension or emulsion in a nontoxic parenterally acceptable diluent or solvent, for example, as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of the vaccine, it is often desirable to slow the absorption of the vaccine from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

The active ingredient can be prepared with carriers that will protect the active ingredient against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Pharmaceutical compositions can be administered with medical devices known in the art. For example, in a preferred embodiment, a pharmaceutical composition of this disclosure can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present disclosure include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation. General considerations in the formulation and/or manufacture of pharmaceutical compositions can be found, for example, in Remington: The Science and Practice of Pharmacy 21^{st} ed., Lippincott Williams & Wilkins, 2005.

The exact amount of the vaccine required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations). In some embodiments, the vaccine is administered as a single dose. In another embodiment, the vaccine is administered as a single dose, followed by 1, 2, 3, 4, 5, 6 7, 8, 9, 10, or more booster doses. In some embodiments, the booster dose does not comprise all of the elements of the vaccine. For example, the booster dose may comprise only the modRNA-RALDH component of the vaccine. In other embodiments, the booster dose may only comprise the modRNA-RALDH and the adjuvant.

In certain embodiments, an effective amount of a vaccine for administration to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of the active ingredient per unit dosage form.

In certain embodiments, the vaccine may be administered parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and from about 1 mg/kg to about 25 mg/kg, of subject body weight per day, one or more times a day, to obtain the desired therapeutic effect.

In certain embodiments, the vaccine may be administered orally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that the vaccines, as described herein, can be administered in combination with one or more additional therapeutically active agents. The vaccines can be administered in combination with additional therapeutically active agents that improve their bioavailability, reduce and/or modify their metabolism, inhibit their excretion, and/or modify their distribution within the body. It will also be appreciated that the therapy employed may achieve a desired effect for the same disorder (for example, a compound can be administered in combination with an anti-cancer agent, etc.), and/or it may achieve different effects (e.g., control of adverse side effects, e.g., emesis controlled by an anti-emetic).

The vaccine can be administered concurrently with, prior to, or subsequent to one or more additional therapeutically active agents. In general, each agent will be administered at a dose and/or on a time schedule determined for that agent. In will further be appreciated that the additional therapeutically active agent utilized in this combination can be administered together in a single composition or administered separately in different compositions. The particular combination to employ in a regimen will take into account compatibility of the active ingredient with the additional therapeutically active agent and/or the desired therapeutic effect to be achieved. In general, it is expected that additional therapeutically active agents utilized in combination be utilized at levels that do not exceed the levels at which they are utilized individually. In some embodiments, the levels utilized in combination will be lower than those utilized individually.

Exemplary additional therapeutically active agents include, but are not limited to, cancer therapies, antibiotics, anti-viral agents, anesthetics, anti-coagulants, inhibitors of an enzyme, steroidal agents, steroidal or non-steroidal anti-inflammatory agents, antihistamine, immunosuppressant agents, anti-neoplastic agents, antigens, additional vaccines, antibodies, decongestant, sedatives, opioids, pain-relieving agents, analgesics, anti-pyretics, hormones, prostaglandins, progestational agents, anti-glaucoma agents, ophthalmic agents, anti-cholinergics, anti-depressants, anti-psychotics, hypnotics, tranquilizers, anti-convulsants/anti-epileptics (e.g., Neurontin, Lyrica, valproates (e.g., Depacon), and other neurostabilizing agents), muscle relaxants, anti-spasmodics, muscle contractants, channel blockers, miotic agents, anti-secretory agents, anti-thrombotic agents, anticoagulants, anti-cholinergics, β-adrenergic blocking agents, diuretics, cardiovascular active agents, vasoactive agents, vasodilating agents, anti-hypertensive agents, angiogenic agents, modulators of cell-extracellular matrix interactions (e.g., cell growth inhibitors and anti-adhesion molecules), or inhibitors/intercalators of DNA, RNA, protein-protein interactions, protein-receptor interactions, etc. Therapeutically active agents include small organic molecules such as drug compounds (e.g., compounds approved by the Food and Drugs Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNA, RNAs, nucleotides, nucleosides, oligonucleotides, antisense oligonucleotides, lipids, hormones, vitamins, minerals, and cells.

In certain embodiments, the additional therapeutic agent is a cancer therapy. Cancer therapies include, but are not limited to, surgery and surgical treatments, radiation therapy, and administration of additional therapeutic cancer agents (e.g., biotherapeutic and chemotherapeutic cancer agents).

Exemplary biotherapeutic cancer agents include, but are not limited to, interferons, cytokines (e.g., tumor necrosis factor, interferon α, interferon γ), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies (e.g. HERCEPTIN (trastuzumab), T-DM1, AVASTIN (bevacizumab), ERBITUX (cetuximab), VECTIBIX (panitumumab), RITUXAN (rituximab), BEXXAR (tositumomab)).

Exemplary chemotherapeutic cancer agents include, but are not limited to, anti-estrogens (e.g. tamoxifen, raloxifene, and megestrol), LHRH agonists (e.g. goserelin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (Abraxane), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonucleotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g., 1-methyl-4-phenylpyridinium ion), cell cycle inhibitors (e.g. staurosporine), actinomycin (e.g., actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS 690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

In certain embodiments, the additional pharmaceutical agent is an immunotherapy. In certain embodiments, the immunotherapy is useful in the treatment of a cancer. Exemplary immunotherapies include, but are not limited to, T-cell therapies, interferons, cytokines (e.g., tumor necrosis factor, interferon α, interferon γ), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies. In certain embodiments, the immunotherapy is a T-cell therapy. In certain embodiments, the T-cell therapy is chimeric antigen receptor T cells (CAR-T). In certain embodiments, the immunotherapy is an antibody. In certain embodiments, the antibody is an anti-PD-l antibody, an anti-PD-L1 antibody, an anti-CTLA-4 antibody, an anti-TIM3 antibody, an anti-OX40 antibody, an anti-GITR antibody, an anti-LAG-3 antibody, an anti-CD137 antibody, an anti-CD27 antibody, an anti-CD28 antibody, an anti-CD28H antibody, an anti-CD30 antibody, an anti-CD39 antibody, an anti-CD40 antibody, an anti-CD47 antibody, an anti-CD48 antibody, an anti-CD70 antibody, an anti-CD73 antibody, an anti-CD96 antibody, an anti-CD160 antibody, an anti-CD200 antibody, an anti-CD244 antibody, an anti-ICOS antibody, an anti-TNFRSF25 antibody, an anti-TMIGD2 antibody, an anti-DNAM1 antibody, an anti-BTLA antibody, an anti-LIGHT antibody, an anti-TIGIT antibody, an anti-VISTA antibody, an anti-HVEM antibody, an anti-Siglec antibody, an anti-GALI antibody, an anti-GAL3 antibody, an anti-GAL9 antibody, an anti-BTNL2 (butrophylins) antibody, an anti-B7-H3 antibody, an anti-B7-H4 antibody, an anti-B7-H5 antibody, an anti-B7-H6 antibody, an anti-KIR antibody, an anti-LIR antibody, an anti-ILT antibody, an anti-MICA antibody, an anti-MICB antibody, an anti-NKG2D antibody, an anti-NKG2A antibody, an anti-TGFβ antibody, an anti-TGFβR antibody, an anti-CXCR4 antibody, an anti-CXCL12 antibody, an anti-CCL2 antibody, an anti-IL-10 antibody, an anti-IL-13 antibody, an anti-IL-23 antibody, an anti-phosphatidylserine antibody, an anti-neuropilin antibody, an anti-GalCer antibody, an anti-HER2 antibody, an anti-VEGFA antibody, an anti-VEGFR antibody, an anti-EGFR antibody, or an anti-Tie2 antibody. In certain embodiments, the antibody is pembrolizumab, nivolumab, pidilizumab, ipilimumab, tremelimumab, durvalumab, atezolizumab, avelumab, PF-06801591, utomilumab, PDR001, PBF-509, MGB453, LAG525, AMP-224, INCSHR1210, INCAGN1876, INCAGN1949, samalizumab, PF-05082566, urelumab, lirilumab, lulizumab, BMS-936559, BMS-936561, BMS-986004, BMS-986012, BMS-986016, BMS-986178, IMP321, IPH2101, IPH2201, varilumab, ulocuplumab, monalizumab, MEDI0562, MEDI0680, MEDI1873, MEDI6383, MEDI6469, MEDI9447, AMG228, AMG820, CC-90002, CDX-1127, CGEN15001T, CGEN15022, CGEN15029, CGEN15049, CGEN15027, CGEN15052, CGEN15092, CX-072, CX-2009, CP-870893, lucatumumab, dacetuzumab, Chi Lob 7/4, RG6058, RG7686, RG7876, RG7888, TRX518, MK-4166, MGA271, IMC-CS4, emactuzumab, trastuzumab, pertuzumab, obinutuzumab, cabiralizumab, margetuximab, enoblituzumab, mogamulizumab, panitumumab, carlumab, bevacizumab, rituximab, or cetuximab.

In certain embodiments, the vaccines or pharmaceutical compositions described herein can be administered in combination with an anti-cancer therapy including, but not limited to, surgery, radiation therapy, and transplantation (e.g., stem cell transplantation, bone marrow transplantation).

In other embodiments, the additional therapeutically active agent is an anti-inflammatory agent. Exemplary anti-inflammatory agents include, but are not limited to, aspirin; ibuprofen; ketoprofen; naproxen; etodolac (LODINE®); COX-2 inhibitors such as celecoxib (CELEBREX®), rofecoxib (VIOXX®), valdecoxib (BEXTRA®), parecoxib, etoricoxib (MK663), deracoxib, 2-(4-ethoxy-phenyl)-3-(4-methanesulfonyl-phenyl)-pyrazolo[1,5-b]pyridazine, 4-(2-oxo-3-phenyl-2,3-dihydrooxazol-4-yl)benzenesulfonamide, darbufelone, flosulide, 4-(4 cyclohexyl-2-methyl-5-oxazolyl)-2-fluorobenzenesulfonamide), meloxicam, nimesulide, 1-Methylsulfonyl-4-(1,1-dimethyl-4-(4-fluorophenyl)cyclopenta-2,4 dien-3-yl)benzene, 4-(1,5-Dihydro-6-fluoro-7-methoxy-3-(trifluoromethyl)-(2)-benzothiopyrano(4,3-c)pyrazol-1-yl)benzenesulfonamide, 4,4-dimethyl-2-phenyl-3-(4-methylsulfonyl)phenyl)cyclo-butenone, 4-Amino-N-(4-(2-fluoro-5-trifluoromethyl)-thiazol-2-yl)-benzene sulfonamide, 1-(7-tert-butyl-2,3-dihydro-3,3-dimethyl-5-benzo-furanyl)-4-cyclopropyl butan-1-one, or their physiologically acceptable salts, esters or solvates; sulindac (CLINORIL®); diclofenac (VOLTAREN®); piroxicam (FELDENE®); diflunisal (DOLOBID®), nabumetone (RELAFEN®), oxaprozin (DAYPRO®), indomethacin (INDOCIN®); or steroids such as PEDIAPED® prednisolone sodium phosphate oral solution, SOLU-MEDROL® methylprednisolone sodium succinate for injection, PRELONE® brand prednisolone syrup.

Further examples of anti-inflammatory agents include naproxen, which is commercially available in the form of EC-NAPROSYN® delayed release tablets, NAPROSYN®, ANAPROX® and ANAPROX® DS tablets and NAPROSYN® suspension from Roche Labs, CELEBREX® brand of celecoxib tablets, VIOXX® brand of rofecoxib, CELESTONE® brand of betamethasone, CUPRAMINE® brand penicillamine capsules, DEPEN® brand titratable penicillamine tablets, DEPO-MEDROL brand of methylprednisolone acetate injectable suspension, ARAVA™ leflunomide tablets, AZULFIDIINE EN-Tabs® brand of sulfasalazine delayed release tablets, FELDENE® brand piroxicam capsules, CATAFLAM® diclofenac potassium tablets, VOLTAREN® diclofenac sodium delayed release tablets, VOLTAREN®-XR diclofenac sodium extended release tablets, or ENBREL® etanercept products.

### Methods of Use and Treatment

Further provided are methods of using the modRNA as described herein. For example, in one aspect, provided is a method of treating a disease, disorder, or condition selected from the group consisting of proliferative disease (e.g., cancer, benign tumors), autoimmune disease, and infectious disease (e.g., bacterial infections, viral infections) comprising administering an effective amount of a modRNA of the present disclosure to a subject in need thereof. In some embodiments, the treatment induces an antigen-specific immune response, such as a T cell and/or B cell response. In other embodiments, the treatment induces an antigen-specific tolerogenic immune response; e.g., reduces a T and/or B cell response.

In certain embodiments, the modRNA of the present disclosure is useful in the treatment of a proliferative disease. Exemplary proliferative diseases include, but are not limited to, cancers and benign neoplasms. In certain embodiments, the proliferative disease is cancer. Exemplary cancers include, but are not limited to, acoustic neuroma, adenocarcinoma, adrenal gland cancer, anal cancer, angiosarcoma (e.g., lymphangiosarcoma, lymphangio-endotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarcinoma), Ewing sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenström's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above, e.g., mixed leukemia lymphoma (MLL); and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendocrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic adenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva). In certain embodiments, the cancer is a mucosal cancer, such as colon cancer, head and neck squamous cell carcinoma, lung cancer, cervical cancer, and gastrointestinal cancer. Examples of gastrointestinal cancers include, but are not limited to, esophageal cancer, stomach (gastric) cancer, and pancreatic cancer.

In certain embodiments, the modRNA of the present disclosure is useful in the treatment of an autoimmune disease. Exemplary autoimmune diseases include, but are not limited to, arthritis (e.g., including rheumatoid arthritis, spondyloarthropathies, gouty arthritis, degenerative joint diseases such as osteoarthritis, systemic lupus erythematosus, Sjogren's syndrome, ankylosing spondylitis, undifferentiated spondylitis, Behcet's disease, haemolytic autoimmune anaemias, multiple sclerosis, amyotrophic lateral sclerosis, amylosis, acute painful shoulder, psoriatic, and juvenile arthritis), asthma, atherosclerosis, osteoporosis, bronchitis, tendonitis, bursitis, skin condition (e.g., psoriasis, eczema, burns, dermatitis, pruritus (itch)), enuresis, eosinophilic disease, gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), and disorders ameliorated by a gastroprokinetic agent (e.g., ileus, postoperative ileus and ileus during sepsis; gastroesophageal reflux disease (GORD, or its synonym GERD); eosinophilic esophagitis, gastroparesis such as diabetic gastroparesis; food intolerances and food allergies and other functional bowel disorders, such as non-ulcerative dyspepsia (NUD) and non-cardiac chest pain (NCCP, including costo-chondritis)).

In certain embodiments, the modRNA of the present disclosure is useful in the treatment or prevention of an infectious disease (e.g., bacterial infection, viral infection). In certain embodiments, the modRNA is useful in treating or preventing a bacterial infection (e.g., Chlamydia). In certain embodiments, the modRNA is useful in treating or preventing a viral infection (e.g., HIV-1).

### Kits

Still further contemplated herein are pharmaceutical packs and/or kits. Pharmaceutical packs and/or kits provided may comprise a provided composition and a container (e.g., a vial, ampoule, bottle, syringe, and/or dispenser package, or other suitable container). In some embodiments, provided kits may optionally further include a second container comprising a suitable aqueous carrier for dilution or suspension of the provided composition for preparation of administration to a subject. In some embodiments, contents of provided formulation container and solvent container combine to form at least one unit dosage form.

Optionally, a single container may comprise one or more compartments for containing a provided composition, and/or appropriate aqueous carrier for suspension or dilution. In some embodiments, a single container can be appropriate for modification such that the container may receive a physical modification so as to allow combination of compartments and/or components of individual compartments. For example, a foil or plastic bag may comprise two or more compartments separated by a perforated seal which can be broken so as to allow combination of contents of two individual compartments once the signal to break the seal is generated. A pharmaceutical pack or kit may thus comprise such multi-compartment containers including a provided composition and appropriate solvent and/or appropriate aqueous carrier for suspension.

Optionally, instructions for use are additionally provided in such kits of the invention. Such instructions may provide, generally, for example, instructions for dosage and administration. In other embodiments, instructions may further provide additional detail relating to specialized instructions for particular containers and/or systems for administration. Still further, instructions may provide specialized instructions for use in conjunction and/or in combination with additional therapy.

### EXAMPLES

In order that the invention described herein may be more fully understood, the following examples are set forth. The synthetic examples described in this application are offered to illustrate the compounds and methods provided herein and are not to be construed in any way as limiting their scope.

### Example 1: In Vitro Effects of modRNA-RALDH2

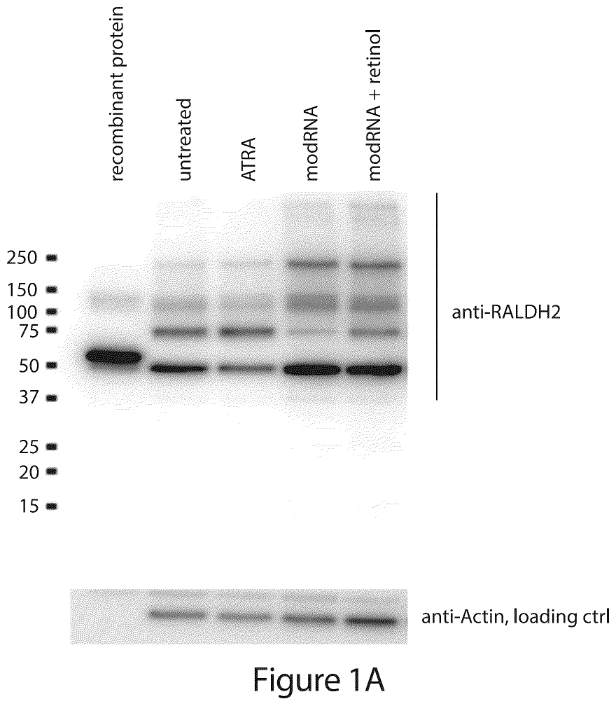

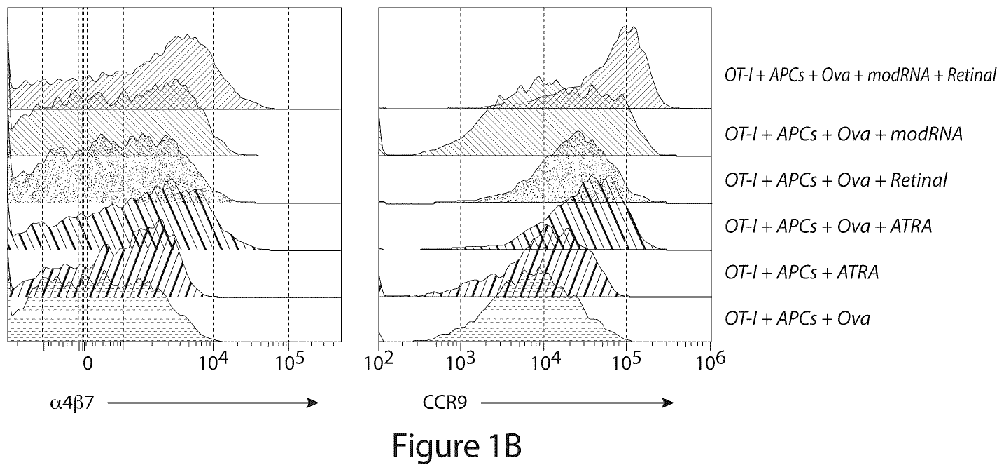

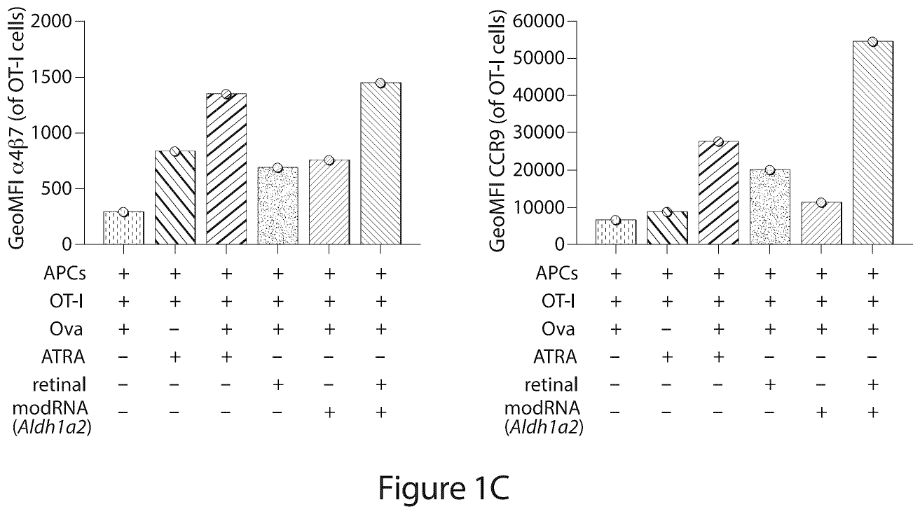

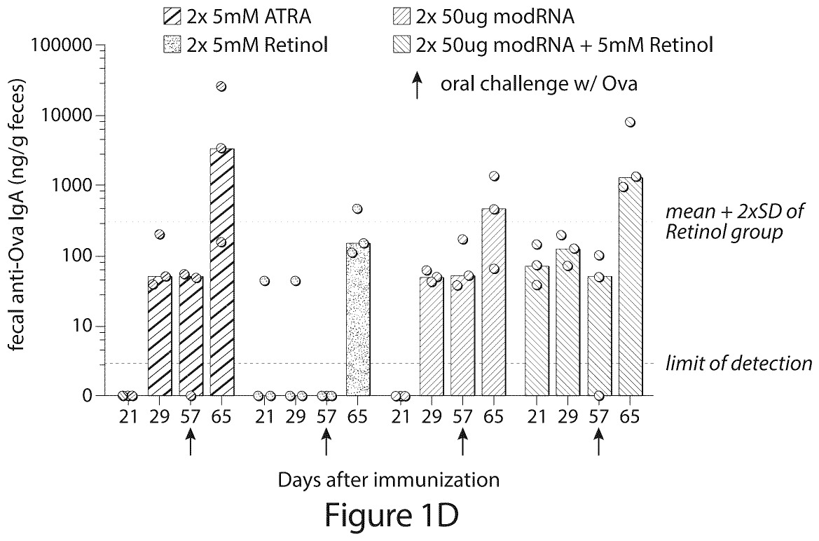

The in vitro effects of modRNA-RALDH2 on RALDH2 expression were examined. Bone marrow-derived dendritic cells (DCs) were cultivated for 48 hours in the presence of 10 nM ATRA, 20 μg modRNA-RALDH2 (modRNA), 20 μg modRNA-RALDH2 plus 1 μM retinol (mod RNA+retinol), or left untouched (untreated). The resulting expression of RALDH2 was detected using a Western blot, and a recombinant human RALDH2 sequence having an N-terminal his-tag was used as a positive control protein. The results, which indicate that modRNA-RALDH2 and modRNA-RALDH2 with retinol induce RALDH2 expression in DCs, are shown in FIG. 1A. Note that the recombinant human RALDH2 protein's his-tag accounts for the apparent size difference seen on the Western blot. The modRNA-RALDH was encapsulated.

The ability of modRNA to induce expression of two gut-homing receptors, α4β7 and CCR9, was also examined in vitro. Purified ovalbumin-specific transgenic CD8⁺ T cells (OT-I) were cultured with wild-type splenocytes (APCs) and the supplements indicated in FIGS. 1B-1C. Flow cytometry (FIG. 1B) and the geometric mean of the mean fluorescent intensity (GeoMFI; FIG. 1C) indicate that the modRNA-RALDH2 transfected DCs induce upregulation of the two gut-homing receptors to the same extent as free ATRA, demonstrating that modRNA-RALDH-exposed DCs have the capacity to activate T cells and provide them with ATRA, which results in a signal to upregulate mucosal homing receptors.

### Example 2: In Vivo Effects of modRNA-RALDH2

Wild-type C57BL/6J mice were randomly divided into groups (each group housed separately) and immunized subcutaneously at day 0 with 100 μg ovalbumin (Ova) and 5 μM CpG class B. At days 0 and 2, the corresponding group of mice received 5 mM ATRA, 5 mM ATRA, 5 mM retinol, 50 μg modRNA, or 50 μg modRNA plus 5 mM retinol, administered subcutaneously at the same site of injection as the Ova/CpG. Note that the modRNA was encapsulated. Fecal samples were collected for the duration of the experiment and assessed for titers of anti-Ova IgA-specific antibodies. All mice were orally challenged by gavage with 10 mg of Ova at day 57. As shown in FIG. 1D, the use of modRNA-RALDH2 in a prime-boost vaccine protocol was sufficient to induce sustained and stable antigen-specific mucosal immunity for at least two months.

### REFERENCES

1. Belyakov I M, Ahlers J D. The Journal of Immunology. 183(11):6883. (2009)
2. Pulendran B, Ahmed R. Nature Immunology. 12(6):509. (2011)
3. Fletcher S M, McLaws M-L, Ellis J T. J Public Health Res. 2(1):42. (2013)
4. Mora J R et al. Nature. 424(6944):88. (2003)
5. Stary G et al. Science. 348(6241):aaa8205. (2015)
6. Azizi A, Kumar A, Diaz-Mitoma F, Mestecky J. PLoS Pathog. 6(11):e1001147. (2010)
7. Nizard M et al. Hum Vaccin Immunother. 10(8):2175. (2014)
8. Wu H Y, Abdu S, Stinson D, Russell M W. Infection and Immunity. 68(10):5539. (2000)
9. Iwata M et al. Immunity. 21(4):527. (2004)
10. Mora J R et al. The Journal of Experimental Medicine. 201(2):303. (2005)
11. Mora J R et al. Science. 314(5802):1157. (2006)
12. Hammerschmidt S I et al. J. Clin. Invest. 121(8):3051. (2011)
13. Nizard M et al. Nat Commun. 8:15221. (2017)

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

1-160. (canceled)

161. A method of inducing an antigen-specific immune response in the gastrointestinal tract of a subject, the method comprising administering a therapeutically effective amount of a mucosal vaccine to the subject to produce the antigen-specific immune response,
wherein the mucosal vaccine comprises:
(a) an antigen from a mucosal pathogen; and
(b) a messenger ribonucleic acid (mRNA) polynucleotide comprising an open reading frame (ORF) encoding a retinaldehyde dehydrogenase (RALDH) protein, wherein the mRNA polynucleotide has been modified such that at least 50% of the uridine residues are pseudouridine, and at least 50% of the cytidine residues are 5-methylcytidine.

162. The method of claim 161, wherein the vaccine is administered to the subject subcutaneously or intramuscularly.

163. The method of claim 161, wherein the antigen-specific immune response is a T cell response, a B cell response, or a T cell and a B cell response.

164. The method of claim 163, wherein the B cell response comprises secretion of IgA.

165. The method of claim 163, wherein the antigen-specific immune response comprises activated T cells or activated B cells or both expressing α4β7 and CCR9.

166. The method of claim 161, wherein the subject is a human or a non-human mammal.

167. The method of claim 161, wherein 100% of the uridine residues are pseudouridine; and 100% of the cytidine residues are 5-methylcytidine.

168. The method of claim 161, wherein the RALDH protein is selected from the group consisting of retinaldehyde dehydrogenase 1 (RALDH1) protein, retinaldehyde dehydrogenase 2 (RALDH2) protein, and retinaldehyde dehydrogenase 3 (RALDH3) protein.

169. The method of claim 168, wherein the RALDH1 protein comprises an amino acid sequence having at least 90% identity to SEQ ID NO: 4; the RALDH2 protein comprises an amino acid sequence having at least 90% identity to SEQ ID NO:5; and the RALDH3 protein comprises an amino acid sequence having at least 90% identity to SEQ ID NO: 6.

170. The method of claim 168, wherein the RALDH protein is RALDH2.

171. The method of claim 170, wherein the RALDH2 protein is a human RALDH2 protein.

172. The method of claim 161, wherein the mRNA is codon-optimized for expression in a human.

173. The method of claim 161, wherein the ORF encodes two RALDH proteins, each selected from the group consisting of: retinaldehyde dehydrogenase 1 (RALDH1) protein, retinaldehyde dehydrogenase 2 (RALDH2) protein, and retinaldehyde dehydrogenase 3 (RALDH3) protein.

174. The method of claim 161, wherein the mucosal vaccine further comprises retinal, retinol, β-carotene, or a combination thereof.

175. The method of claim 161, wherein the mucosal vaccine is formulated as a nanoparticle, microparticle, hydrogel, or liposome.

176. The method of claim 161, wherein the mucosal pathogen is a viral pathogen, a bacterial pathogen, a fungal pathogen, or a combination thereof.

177. The method of claim 176, wherein the bacterial pathogen is selected from the group consisting of Bacillus species, Bartonella species, Bordetella species, Borrelia species, Campylobacter species, Chlamydia species, Chlamydophila species, Clostridium species, Corynebacterium species, Enterococcus species, Escherichia species, Francisella species, Haemophilus species, Helicobacter species, Legionella species, Leptospira species, Listeria species, Mycobacterium species, Mycoplasma species, Neisseria species, Pseudomonas species, Rickettsia species, Salmonella species, Shigella species, Staphylococcus species, Streptococcus species, Treponema species, Ureaplasma species, Vibrio species, and Yersinia species.

178. The method of claim 176, wherein the viral pathogen is selected from the group consisting of: Aichi virus, Astrovirus, Australian bat lyssavirus, Banna virus, Barmah forest virus, Bunyamwera virus, Bunyavirus, Cercopithecine herpesvirus, Chandipura virus, Chikungunya virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, Hantaan virus, Hendra virus, Hepatitis A virus, Hepatitis B virus, Hepatitis C virus, Hepatitis E virus, Hepatitis delta virus, Human adenovirus, Human astrovirus, Human coronavirus, Human cytomegalovirus, Human enterovirus 68, 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16,18, Human parainfluenza, Human parvovirus B19, Human respiratory syncytial virus, Human rhinovirus, Human T-lymphotropic virus, Human torovirus, Influenza A virus, Influenza B virus, Influenza C virus, JC polyomavirus, Japanese encephalitis virus, Junin arenavirus, KI Polyomavirus, Kunjin virus, Lagos bat virus, Lake Victoria Marburgvirus, Langat virus, Lassa virus, Lordsdale virus, Louping ill virus, Lymphocytic choriomeningitis virus, Machupo virus, Mastadenovirus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norovirus (Norwalk virus), O'nyong-nyong virus, Orf virus, Oropouche virus, Pichinde virus, Poliovirus, Punta toro phlebovirus, Puumala virus, Rabies virus, Rift valley fever virus, Ross river virus, Rotavirus A, Rotavirus B, Rotavirus C, Rubella virus, Sagiyama virus, Salivirus A, Sandfly fever Sicilian virus, Sapporo virus, Semliki forest virus, Seoul virus, Sindbis virus, Southampton virus, St. Louis encephalitis virus, Tick-borne powassan virus, Toscana virus, Uukuniemi virus, Varicella-zoster virus, Venezuelan equine encephalitis virus, Vesicular stomatitis virus, Western equine encephalitis virus, West Nile virus, Yellow fever virus, and Zika virus.

179. A method of inducing an antigen-specific immune response in the gastrointestinal tract of a human subject, the method comprising administering a therapeutically effective amount of a mucosal vaccine to the human subject to produce the antigen-specific immune response,
wherein the mucosal vaccine comprises:
(a) an antigen from a mucosal pathogen;
(b) a messenger ribonucleic acid (mRNA) polynucleotide comprising an open reading frame (ORF) encoding a human retinaldehyde dehydrogenase 2 (RALDH2) protein, wherein the human RALDH2 protein comprises an amino acid sequence having at least 90% identity to SEQ ID NO: 5, wherein the mRNA polynucleotide has been modified such that 100% of the uridine residues are pseudouridine, and 100% of the cytidine residues are 5-methylcytidine, and wherein the mRNA is encapsulated in a lipid nanoparticle; and
(c) retinol.

180. The method of claim 179, wherein the mucosal vaccine further comprises an adjuvant.


SEQUENCE LISTING (6 sequences)

SEQ ID NO: 1 - RNA, 1506 nt, synthetic construct
  misc_feature 1..1506: note = Synthetic polynucleotide
  source 1..1506: mol_type = other RNA
atgtcttcgcctgcacaacctgcagtccctgccccactggccgacttgaagattcaacataccaagatcttcataaacaatgaatggcacaattcagtgagcggcaagaaatttccagttcttaaccctgcaactgaggaggtcatctgccacgtggaagaaggggacaaggctgatgttgacaaagctgtgaaggctgcaagacaggctttccagattggctctccatggcgcaccatggatgcttcagagaggggccgcctgctgaacaagctggctgacttaatggagagagatcgtctgctgctggctacaatggaggcactcaatggtgggaaagtctttgccaatgcatacttgtcggatttaggaggctgcataaaagcattaaagtactgtgcaggctgggctgacaagattcatggtcaaacaataccaagtgatggagacattttcacttatacaagacgtgaacctattggagtgtgtggccaaatcatcccctggaattttccaatgctcatgttcatttggaagataggccctgcccttagctgtgggaataccgtggttgtcaagccagcagagcaaactcctctcacggctcttcacctggcatctttaataaaagaggcagggtttcctcctggcgtggtaaacattgtccctggttatgggccaactgcaggggcagccatctcctctcacatggatgtcgacaaggtggccttcactggatcaacacaggttggcaagttaatcaaggaagctgcagggaaaagcaatctgaagagagtcaccctggagctggggggaaagagcccttgcattgtgtttgcagatgccgacttggacattgctgttgagtttgcacaccatggagtgttttatcatcaaggccaatgttgtgtcgcagcatcccggatttttgttgaggagtcagtttatgatgagtttgtgaaaaggagtgttgagcgagctaagaaatatgttcttggaaatcctctgaccccaggaataaatcaaggccctcagattgacaaggaacaacatgataaaatactcgatctcattgagagtgggaagaaagaaggagccaaactggagtgtggtggaggacgctgggggaacaaaggcttctttgtgcagcccacagtgttctccaacgtgactgatgagatgcgcattgccaaagaggagatatttggaccagtgcaacaaatcatgaagtttaagtctgtagatgatgtgatcaagagagcaaacaatactacctatggtttagcagcaggactcttcactaaagacctggataaggccatcactgtgtcatctgctctgcaggctggggtggtgtgggttaactgctatatgatgttgtcagcccagtgccccttcggtggattcaagatgtctggaaatggaagagaactgggtgaacatggtctttatgaatacactgagctcaagacagtcgcaatgaagatatctcagaagaactcctaa

SEQ ID NO: 2 - RNA, 1557 nt, synthetic construct
  misc_feature 1..1557: note = Synthetic polynucleotide
  source 1..1557: mol_type = other RNA
atgacttccagcgagatcgccatgccgggcgaggtgaaggccgaccccgccgcgctcatggcctcgctgcagctcctgccgtcgcccacgcccaacctcgagatcaagtacaccaagatctttattaacaatgaatggcagaactcagagagtgggagagtgttccctgtctgtaatccagccacaggagagcaagtgtgtgaagttcaagaagcagacaaggtggatatagacaaggcagtgcaagcagcccgccttgccttctctcttggttctgtgtggagaaggatggatgcgtctgaaagaggacgtctgttggacaagcttgcagacttggtggaacgggacagggcaactcttgcaactatggaatccctaaatggcggtaagccattcctgcaagctttttacatcgatttgcagggagtcatcaaaaccctgaggtattatgcaggctgggctgataaaattcacggaatgaccattcctgtagatggagactattttactttcacaagacacgagcccattggagtgtgtggacagatcatcccgtggaacttccccctgctgatgttcacctggaaaattgctcccgcattgtgctgtggtaacaccgtggtcatcaaacccgctgagcagacaccgctcagtgcactgtatatgggagccctcatcaaggaggctggctttccacccggagtcgtcaatatcctgccagggtatgggccaacagcaggggcagcaatcgcttctcacatcggcatagacaagattgcattcacaggctctactgaggttggaaagcttattcaagaagcagctggaagaagtaacctgaagagagtgaccctggaactcggagggaagagccctaatattatttttgcagatgctgacttggactacgctgtggagcaggctcaccagggtgtgttcttcaaccaaggccagtgctgcaccgcagggtctcgcatctttgtggaagaatccatctatgaggaatttgtgaaaagaagtgtggaaagggccaagaggcgcattgtggggagcccctttgatcccacaactgagcagggtcctcagatcgataagaaacaatacaacaaagtcctggagcttatccagagcggtgtggctgagggcgccaagctggagtgcggaggcaaagggctgggaaggaagggcttcttcattgagcccaccgtgttctccaacgtcactgatgacatgcggattgccaaggaggagatctttggtcctgttcaagaaattttgaggtttaagactatggatgaagttatagaaagagccaataactcagactttggacttgtagcagctgtcttcactaatgacatcaacaaggctctcatggtatcctccgcaatgcaagctgggacagtttggatcaattgttacaatgccttaaatgcgcagagcccctttggggggttcaagatgtctggaaatgggagagaaatgggtgagtttggcttacgggagtattcagaagtaaagaccgtgacggtgaagatcccccagaagaactcctaa

SEQ ID NO: 3 - RNA, 1539 nt, synthetic construct
  misc_feature 1..1539: note = Synthetic polynucleotide
  source 1..1539: mol_type = other RNA
atggctaccaccaacggggctgtggaaaacggacagccggatgggaaaccgcctgccttgccgcgccccatccgcaacttggaggtcaagttcaccaagatatttatcaacaacgactggcacgaatccaagagtggaagaaagtttgccacatataacccttcaacactagagaaaatatgtgaggtggaagaaggagataagcccgatgtggacaaggctgtggaggccgctcaagctgccttccagcggggatccccgtggcgccggctggatgcactgagcagaggccagttgctgcatcagctggctgaccttgtagaaagggaccgagcgatcctggctactctggagaccatggacaccggcaagccattccttcatgcctttttcgtcgacctggaaggctgtattaagaccttcagatattttgccgggtgggcagacaaaatccagggcaggaccatccccacagatgacaacgttgtgtgcttcaccaggcatgagcccatcggggtgtgtggggccattacaccatggaacttccccctgctgatgctggcctggaaactggctcctgccctgtgctgtgggaacaccgtggtcctgaagccagctgagcagacccctctcacggctctgtacctagcctctctcatcaaagaggtcgggttccctccgggtgtggtgaacattgtaccaggctttgggcccactgtgggagcagcaatttcctcccatccgcagatcaacaagatagccttcaccggctccacagaggttggaaagctggtcagagaagccgcctcccggagcaacctgaagagggtcacactggagctaggaggcaagaacccgtgcatcgtgtgtgcagatgctgacttggacttggccgtcgagtgtgctcaccagggagtgttcttcaaccaaggccagtgctgtacagcggcctccagggtgttcgtggaagagcaggtctacggggagtttgtgaggaggagtgtggagttcgccaagaagaggccagttggagaccccttcgatgccaaaacggagcaggggcctcagatcgaccaaaagcagtttgacaaaatcctcgagctgattgagagtgggaagaaggaaggggccaagctagaatgtggggggtcagccatggaggacagagggctgttcatcaaacccacggtcttctcagatgttacggacaacatgaggattgccaaagaggagattttcggaccagtgcagccgatcctgaagttcaaaaacctggaggaggtgatcaagagagcgaatagcaccgactatggactcacagcagcagtgttcaccaaaaacctggacaaagcactgaagctggctgctgcgctcgagtcggggacagtctggatcaactgctacaatgcattttatgcacaggctccatttggtggcttcaaaatgtctgggaatggcagagaactaggagaatatgctctggctgaatatacagaagtgaaaactgtcaccatcaaactcgaggagaagaacccctga

SEQ ID NO: 4 - protein, 501 aa, Homo sapiens
MSSSGTPDLPVLLTDLKIQYTKIFINNEWHDSVSGKKFPVFNPATEEELCQVEEGDKEDVDKAVKAARQAFQIGSPWRTMDASERGRLLYKLADLIERDRLLLATMESMNGGKLYSNAYLNDLAGCIKTLRYCAGWADKIQGRTIPIDGNFFTYTRHEPIGVCGQIIPWNFPLVMLIWKIGPALSCGNTVVVKPAEQTPLTALHVASLIKEAGFPPGVVNIVPGYGPTAGAAISSHMDIDKVAFTGSTEVGKLIKEAAGKSNLKRVTLELGGKSPCIVLADADLDNAVEFAHHGVFYHQGQCCIAASRIFVEESIYDEFVRRSVERAKKYILGNPLTPGVTQGPQIDKEQYDKILDLIESGKKEGAKLECGGGPWGNKGYFVQPTVFSNVTDEMRIAKEEIFGPVQQIMKFKSLDDVIKRANNTFYGLSAGVFTKDIDKAITISSALQAGTVWVNCYGVVSAQCPFGGFKMSGNGRELGEYGFHEYTEVKTVTVKISQKNS

SEQ ID NO: 5 - protein, 518 aa, Homo sapiens
MTSSKIEMPGEVKADPAALMASLHLLPSPTPNLEIKYTKIFINNEWQNSESGRVFPVYNPATGEQVCEVQEADKADIDKAVQAARLAFSLGSVWRRMDASERGRLLDKLADLVERDRAVLATMESLNGGKPFLQAFYVDLQGVIKTFRYYAGWADKIHGMTIPVDGDYFTFTRHEPIGVCGQIIPWNFPLLMFAWKIAPALCCGNTVVIKPAEQTPLSALYMGALIKEAGFPPGVINILPGYGPTAGAAIASHIGIDKIAFTGSTEVGKLIQEAAGRSNLKRVTLELGGKSPNIIFADADLDYAVEQAHQGVFFNQGQCCTAGSRIFVEESIYEEFVRRSVERAKRRVVGSPFDPTTEQGPQIDKKQYNKILELIQSGVAEGAKLECGGKGLGRKGFFIEPTVFSNVTDDMRIAKEEIFGPVQEILRFKTMDEVIERANNSDFGLVAAVFTNDINKALTVSSAMQAGTVWINCYNALNAQSPFGGFKMSGNGREMGEFGLREYSEVKTVTVKIPQKNS

SEQ ID NO: 6 - protein, 512 aa, Homo sapiens
MATANGAVENGQPDRKPPALPRPIRNLEVKFTKIFINNEWHESKSGKKFATCNPSTREQICEVEEGDKPDVDKAVEAAQVAFQRGSPWRRLDALSRGRLLHQLADLVERDRATLAALETMDTGKPFLHAFFIDLEGCIRTLRYFAGWADKIQGKTIPTDDNVVCFTRHEPIGVCGAITPWNFPLLMLVWKLAPALCCGNTMVLKPAEQTPLTALYLGSLIKEAGFPPGVVNIVPGFGPTVGAAISSHPQINKIAFTGSTEVGKLVKEAASRSNLKRVTLELGGKNPCIVCADADLDLAVECAHQGVFFNQGQCCTAASRVFVEEQVYSEFVRRSVEYAKKRPVGDPFDVKTEQGPQIDQKQFDKILELIESGKKEGAKLECGGSAMEDKGLFIKPTVFSEVTDNMRIAKEEIFGPVQPILKFKSIEEVIKRANSTDYGLTAAVFTKNLDKALKLASALESGTVWINCYNALYAQAPFGGFKMSGNGRELGEYALAEYTEVKTVTIKLGDKNP